SEQUENCE LISTING (51 sequences)

SEQ ID NO: 1 - protein, 21 aa, Human immunodeficiency virus type 1
  MISC_FEATURE (21)..(21): X may be any amino acid, preferably A or P
MEPVDPRLEPWKHPGSQPKTX

SEQ ID NO: 2 - protein, 21 aa, Human immunodeficiency virus type 1
  MISC_FEATURE (21)..(21): X may be any amino acid, preferably A or P
MEPVDPNLEPWNHPGSQPKTX

SEQ ID NO: 3 - protein, 21 aa, Human immunodeficiency virus type 1
  MISC_FEATURE (21)..(21): X maqy be any amino acid, preferably A or P
MEPVDPRLEPWNHPGSQPKTX

SEQ ID NO: 4 - protein, 21 aa, Human immunodeficiency virus type 1
  MISC_FEATURE (21)..(21): X may be any amino acid, preferably A or P
MEPVDPNLEPWKHPGSQPKTX

SEQ ID NO: 5 - protein, 21 aa, Human immunodeficiency virus type 1
MEPVDPRLEPWKHPGSQPKTA

SEQ ID NO: 6 - protein, 21 aa, Human immunodeficiency virus type 1
MEPVDPRLEPWKHPGSQPKTP

SEQ ID NO: 7 - DNA, 63 bp, Human immunodeficiency virus type 1
  misc_feature (61)..(63): nnn may be any codon, preferably encoding A or P
atggagccagtagatcctagactagagccctggaagcatccaggaagtcagcctaagactnnn

SEQ ID NO: 8 - DNA, 63 bp, Human immunodeficiency virus type 1
  misc_feature (61)..(63): nnn may be any codon, preferably encoding A or P
atggagccagtagatcctaatctagagccctggaatcatccaggaagtcagcctaagactnnn

SEQ ID NO: 9 - DNA, 63 bp, Human immunodeficiency virus type 1
  misc_feature (61)..(63): nnn may be any codon, preferably encoding A or P
atggagccagtagatcctagactagagccctggaatcatccaggaagtcagcctaagactnnn

SEQ ID NO: 10 - DNA, 63 bp, Human immunodeficiency virus type 1
  misc_feature (61)..(63): nnn may be any codon, preferably encoding A or P
atggagccagtagatcctaatctagagccctggaagcatccaggaagtcagcctaagactnnn

SEQ ID NO: 11 - DNA, 63 bp, Human immunodeficiency virus type 1
atggagccagtagatcctagactagagccctggaagcatccaggaagtcagcctaagactgct

SEQ ID NO: 12 - DNA, 63 bp, Human immunodeficiency virus type 1
atggagccagtagatcctagactagagccctggaagcatccaggaagtcagcctaagactccc

SEQ ID NO: 13 - protein, 15 aa, Artificial Sequence
  Synthetic Construct
SYGSKKRRQRRRPPQ

SEQ ID NO: 14 - protein, 12 aa, Artificial Sequence
  Synthetic Construct
SYGSKKRRQRRR

SEQ ID NO: 15 - protein, 16 aa, Artificial Sequence
  Synthetic Construct
KALGISYGSKKRRQRR

SEQ ID NO: 16 - protein, 20 aa, Artificial Sequence
  Synthetic Construct
MEPVDPRLEPWKHPGSQPKT

SEQ ID NO: 17 - protein, 20 aa, Artificial Sequence
  Synthetic Construct
MEPVDPKLEPWKHPGSQPRT

SEQ ID NO: 18 - protein, 20 aa, Artificial Sequence
  Synthetic Construct
MEPVDPNLEPWKHPGSQPRT

SEQ ID NO: 19 - protein, 20 aa, Artificial Sequence
  Synthetic Construct
MEPVDPNLEPWNHPGSQPKT

SEQ ID NO: 20 - protein, 20 aa, Artificial Sequence
  Synthetic Construct
MDPVDPSLEPWNHPGSQPKT

SEQ ID NO: 21 - protein, 20 aa, Artificial Sequence
  Synthetic Construct
DPGTVEPKPLHPERKQMPWS

SEQ ID NO: 22 - protein, 20 aa, Artificial Sequence
  Synthetic Construct
SQPKTACTNCYCKKCCFHCQ

SEQ ID NO: 23 - protein, 20 aa, Artificial Sequence
  Synthetic Construct
SQPRTACTSCYCKKCCFHCQ

SEQ ID NO: 24 - protein, 20 aa, Artificial Sequence
  Synthetic Construct
SQPRTACNNCYCKKCCFHCY

SEQ ID NO: 25 - protein, 20 aa, Artificial Sequence
  Synthetic Construct
SQPKTACNKCYCKNCSYHCL

SEQ ID NO: 26 - protein, 20 aa, Artificial Sequence
  Synthetic Construct
SQPKTACNTCYCKKCCYHCQ

SEQ ID NO: 27 - protein, 20 aa, Artificial Sequence
  Synthetic Construct
TCCQKNKCPTKHQCCFSAYC

SEQ ID NO: 28 - protein, 20 aa, Artificial Sequence
  Synthetic Construct
CFHCQVCFMTKALGISYGRK

SEQ ID NO: 29 - protein, 20 aa, Artificial Sequence
  Synthetic Construct
CFHCQVCFITKGLGISYGSK

SEQ ID NO: 30 - protein, 20 aa, Artificial Sequence
  Synthetic Construct
CFHCYACFTRKGLGISYGRK

SEQ ID NO: 31 - protein, 20 aa, Artificial Sequence
  Synthetic Construct
SYHCLVCFQTKGLGISYGRK

SEQ ID NO: 32 - protein, 20 aa, Artificial Sequence
  Synthetic Construct
CYHCQVCFLNKGLGISYGRK

SEQ ID NO: 33 - protein, 20 aa, Artificial Sequence
  Synthetic Construct
QTIKCMGRFHLFGCAYCVKS

SEQ ID NO: 34 - protein, 20 aa, Artificial Sequence
  Synthetic Construct
SYGRKKRRQRRRAHQNSQTH

SEQ ID NO: 35 - protein, 20 aa, Artificial Sequence
  Synthetic Construct
SYGSKKRRQRRRPPQDNQTH

SEQ ID NO: 36 - protein, 20 aa, Artificial Sequence
  Synthetic Construct
SYGRKKRRQRRRAPQDSQTH

SEQ ID NO: 37 - protein, 20 aa, Artificial Sequence
  Synthetic Construct
SYGRKKRRQRRSAPPSSEDH

SEQ ID NO: 38 - protein, 20 aa, Artificial Sequence
  Synthetic Construct
SYGRKKRRQRRSAPPSNGDH

SEQ ID NO: 39 - protein, 20 aa, Artificial Sequence
  Synthetic Construct
QKRHRQHTGRAQYRSRSKRN

SEQ ID NO: 40 - protein, 20 aa, Artificial Sequence
  Synthetic Construct
NSQTHQASLSKQPTSQSRGD

SEQ ID NO: 41 - protein, 20 aa, Artificial Sequence
  Synthetic Construct
CNQTHQVSLSKQPSSQPRGD

SEQ ID NO: 42 - protein, 20 aa, Artificial Sequence
  Synthetic Construct
DSQTHQASLSKQPASQSRGD

SEQ ID NO: 43 - protein, 20 aa, Artificial Sequence
  Synthetic Construct
SSEDHQNLIPKQPLPRTQGD

SEQ ID NO: 44 - protein, 20 aa, Artificial Sequence
  Synthetic Construct
SNGDHQNPISKQPLPQTRGD

SEQ ID NO: 45 - protein, 20 aa, Artificial Sequence
  Synthetic Construct
HSSDTLTGQSPRSAQSNQKQ

SEQ ID NO: 46 - protein, 20 aa, Artificial Sequence
  Synthetic Construct
QSRGDPTGPKESKKKVERET

SEQ ID NO: 47 - protein, 20 aa, Artificial Sequence
  Synthetic Construct
QPRGDPTGPKESKKKVERET

SEQ ID NO: 48 - protein, 20 aa, Artificial Sequence
  Synthetic Construct
QSRGDPTGPTESKKKVERET

SEQ ID NO: 49 - protein, 20 aa, Artificial Sequence
  Synthetic Construct
RTQGDPTGSEESKKKVESKT

SEQ ID NO: 50 - protein, 20 aa, Artificial Sequence
  Synthetic Construct
QTRGDPTGSKESKKEVESKT

SEQ ID NO: 51 - protein, 20 aa, Artificial Sequence
  Synthetic Construct
KDKTGEKPRKGRSTSEQPEV

(19) United States
(12) Patent Application Publication
(10) Pub. No.: US 2006/0104988 A1
(43) Pub. Date: May 18, 2006

(54) Vaccines against hiv-1 tat protein to generate neutralizing antibodies

(76) Inventors: C David Pauza, Baltimore, MD (US); Ilia Tikhonov, Bounie Ridge (BY)

Correspondence Address: INTELLECTUAL PROPERTY / TECHNOLOGY LAW, PO BOX 14329, RESEARCH TRIANGLE PARK, NC, 27709, US

(21) Appl. No.: 10/539,677

(22) PCT Filed: Dec. 18, 2003
(86) PCT No.: PCT/US03/40568

§ 371 Date: Sep. 6, 2005

(30) Foreign Application Priority Data
Dec. 18, 2002 (US) 60434368

(51) Int. Cl. A61K 39/21; C07K 14/16

(52) U.S. Cl. 424/188.1; 530/350

(57) ABSTRACT

The present invention relates to compositions and methods for eliciting a neutralizing antibody response specific to HIV Tat proteins. Also disclosed are vaccines including amino terminus Tat linear epitope peptide fragments having the amino terminus sequence of the HIV Tat protein, optionally conjugated to a carrier protein. The invention relates as well to the nucleotide sequences encoding the peptide fragments, recombinant vectors carrying the sequences, recombinant host cells including either the sequences or vectors, and recombinant peptides. The invention further includes methods for using the isolated, recombinant peptides in vaccines, assays, and for use in therapeutic applications.

### BACKGROUND OF THE INVENTION

1. Field of the Invention

The present invention relates to compositions and methods for eliciting a neutralizing antibody response specific to HIV Tat proteins, and more particularly, to vaccines comprising peptide fragments having the amino terminus sequence of the HIV Tat protein, optionally conjugated to a carrier protein. The invention relates as well to the nucleotide sequences encoding the peptide fragments, recombinant vectors carrying the sequences, recombinant host cells including either the sequences or vectors, and recombinant peptides. The invention further includes methods for using the isolated, recombinant peptides in vaccines, assays, and for use in preventive and therapeutic applications.

2. Description of the Related Art

The human immunodeficiency virus type 1 (HIV-1) Tat protein is required for virus replication and pathogenesis. Tat is produced early in the virus life cycle from a multiply spliced mRNA and is transported back into the cell nucleus, where it interacts with host factors and the TAR region of viral RNA to relieve a block of transcript elongation and increase viral gene expression (reviewed in reference 30). Extracellular Tat has distinct functions that may indirectly promote virus replication and disease (20,21) either through receptor-mediated signal transduction (2, 47) or after internalization and transport to the nucleus (17, 18, 35). These major properties of Tat, i.e., early expression to increase viral gene transcription and indirect effects as an extracellular factor, prompted efforts to develop this whole protein as an HIV-1 vaccine antigen.

The Tat protein is encoded by two exons near the center of the viral genome. The first exon encodes amino acids (aa) 1 to 72, and the second exon encodes aa 73 to 101, although naturally occurring Tat sequences may be up to 113 aa long (30). A mutation in some laboratory isolates (IIIB strains) created an 86-aa version that is sufficient for virus replication in vitro and is the form of Tat studied most often. The Tat protein itself contains several functional subdomains. The amino terminus (aa 1 to 20), cysteine-rich domain (aa 21 to 40), and core region (aa 1 to 48) together constitute the minimal activation domain for transcription in vitro (30). The amino terminal portion of Tat binds cell surface CD26 with high affinity and is believed to be responsible for CD26-mediated immunosuppressive activity (26, 49, 57).

The cysteine-rich domain has homology to chemokines and mediates binding to chemokine receptors (1, 2, 16).

The basic domain of Tat protein (aa 45 to 56), characterized by a high content of lysines and arginines, is required for binding to short RNA transcripts containing the viral transactivation responsive element (14, 15, 54). This basic domain is essential for importing extracellular Tat and also binds to membrane proteins, including the vascular endothelial growth factor receptor and heparan sulfate proteoglycans (54). Free peptide corresponding to the basic domain of Tat translocates through the cellular membrane and accumulates in the nucleus (42,53). Chimeric or modified proteins that include the Tat basic domain sequence readily enter a variety of cell types (18, 45). The basic domain may also mediate toxin like properties of Tat, including neuronal toxicity (37), and it appears to signal through cyclic nucleoside phosphodiesterase 4 to alter cyclic AMP levels (47).

The function of the C terminus of Tat is largely unknown, but it contains an RGD motif that is know to mediate Tat binding to cell surface integrins (5, 10).

An important reason for developing vaccines against Tat is to control the toxic properties of this protein. Tat suppresses mitogen, alloantigen, and antigen induced lymphocyte proliferation in vitro (8, 26, 49, 52) by stimulating suppressive levels of alpha interferon (58) and by inducing apoptosis in activated lymphocytes (56). Apoptosis may be triggered directly, upon induction of caspase pathways (6, 34), or indirectly, through increased expression of CD95 or TRAIL in monocytes/macrophages (31, 56, 60). In vivo Tat may alter immunity by upregulating interleukin 10 and reducing interleukin 12 production (4, 29) or through its ability to increase chemokine receptor expression (28, 46, 51, 55). Patients infected with HIV-1 often develop antibody responses to Tat protein that may be correlated with clinical status (38, 39, 59). In the past, patient sera (39) and sera from immunized mice (11) or macaques (48) were used in rough mapping of Tat epitopes, but the breadth of response for different viral sequences has not been reported.

The potential value of Tat as a vaccine antigen is controversial. Published reports of complete (12) or partial (22, 36) protection against virus challenge in macaques contrast with studies showing no protection effects (3, 48). Generalizations are elusive, partly because each group used different animal models, antigens, and vaccination protocols and also because there are no standardized assays for Tat immune responses. Further, previous studies did not define the mechanisms for neutralizing extracellular Tat or epitopes that elicit neutralizing antibodies.

Existing Tat vaccines are whole protein or protein fragment preparations delivered either as soluble protein or encoded as DNA constructs. At present, no specific regions of Tat have been identified as preferred targets for vaccination, principally because data are lacking about the function of individual sequences.

Heretofore, part of the rationale for Tat vaccines is that the viral protein has toxic and immunosuppressive effects in vivo. Accordingly, vaccines based on whole protein antigens utilize chemical modification or genetic deletion of cysteine residues, to reduce toxicity, while still retaining the whole antigen to elicit the maximum antibody response. Potential toxicity for Tat vaccines is addressed through either chemical modification of cysteine residues in the whole Tat protein, or genetic deletion of these residues in DNA constructs. These efforts reflect the desire to retain sequences from the “basic region” of Tat that are believed critical for internalization and biological activities.

U.S. Pat. No. 5,606,026 discloses human IgM antibodies which are reactive to a specific peptide fragment of HIV-1 Tat. U.S. Pat. No. 6,132,721 discloses a 15 amino acid amino terminal peptide fragment of Tat and chemical derivatization of HIV proteins to reduce their toxic activity. U.S. Pat. No. 6,193,981 discloses compositions comprising amino terminal peptide fragments of Tat. U.S. Pat. No. 6,319,666 discloses a method of stimulating cytotoxic T-cell lymphocytes using Tat protein. But to date there has been no discussion relating to vaccines that elicit the production of antisera capable of neutralizing the biological activity of Tat internalization in T cells.

Thus, it would be advantageous to develop a vaccine that does not suffer from the short comings of the full Tat protein vaccines and elicits antibodies capable of neutralizing Tat thereby reducing the negative effects inherent in the increase of Tat in HIV infected patients.

### SUMMARY OF THE INVENTION

The present invention relates to enhancing levels of antibodies against Tat proteins thereby reducing internalization of Tat into T cells by administering a composition that elicits production of antibodies that recognize and bind linear epitopes of the Tat protein, wherein the linear epitope is preferably positioned on the amino terminus.

In another aspect, the present invention relates to therapeutic compositions comprising at least one peptide having an amino acid sequence of the amino terminus of the Tat protein conjugated to a carrier protein, wherein the peptides elicits production of B-cell epitope binding antibodies that inhibit entry of Tat into T cell. Preferably, the carrier protein is a viral protein, and more preferably a HIV viral protein or fragment thereof.

In another aspect, the invention relates to a therapeutic vaccine comprising at least one peptide having at least 15 amino acid residues from the amino terminus region of Tat, wherein the amino acid sequence comprises at least amino acid residue 1, 7 and 12 of the amino terminus of Tat. Preferably, the peptide comprises from about 15 to about 21 amino acid residues from the amino terminus of the Tat protein.

Another aspect of the present invention relates to a method to induce production of neutralizing Tat antibodies that inhibit internalization of Tat into T-cells, the method comprising:

administering to a subject a vaccine comprising at least one peptide having at least 15 amino acid residues from the amino terminus region of Tat conjugated to a viral carrier protein, wherein the amino acid sequence comprises at least amino acid residue 1, 7 and 12 in an effective amount to induce production of neutralizing Tat antibodies.

The therapeutic compositions of the present invention may be administered in combination with at least one antiviral agent. The antiviral agent may include any agent that inhibits entry into a cell or replication therein of an infectious virus, and specifically retroviruses, such as HIV viruses. The antiviral agents include, but are not limited to nucleoside RT inhibitors, CCR5 inhibitors/antagonists, viral entry inhibitors and their functional analogs.

In still another aspect, the present invention relates to a therapeutic method of combating an HIV virus infection, comprising:

administering to a patient a composition comprising an effective amount of at least one peptide having at least about 15 to about 21 amino acid residues from the amino terminus region of Tat, preferably conjugated to a carrier protein, wherein the amino acid sequence comprises at least amino acid residue 1, 7 and 12 thereby inducing production of cross-reactive antibodies for Tat neutralization in multiple clades.

In yet another aspect, the present invention relates to a method of maintaining durable viral control of HIV, the method comprising:

administering at least one isolated peptide or a nucleotide sequence that expresses the peptide, wherein the at least one peptide comprises about 15 to about 21 amino acid residues from the amino terminus region of Tat, optionally conjugated to a carrier protein, wherein the amino acid sequence comprises at least amino acid residue 1, 7 and 12 of HIV Tat.

In still a further aspect, the present invention relates to a method of preventing HIV in a subject potentially exposed to the HIV, the method comprising:

administering at least one isolated peptide having at least about 15 to about 21 amino acid residues from the amino terminus region of HIV Tat, conjugated to a viral carrier protein, wherein the amino acid sequence comprises at least amino acid residue 1, 7 and 12 of the Tat protein.

Another aspect of the present invention provides polynucleotide sequences having a nucleotide sequence encoding a peptide having at least about 15 to about 21 amino acid residues from the amino terminus region of HIV Tat, optionally linked to a nucleotide sequence encoding for a viral carrier protein, wherein the amino acid sequence comprises at least amino acid residue 1, 7 and 12 from the amino terminus region of Tat.

The polynucleotides can be included in an expression vector and used for expressing the isolated peptides and optional viral protein.

Also, the present invention includes antibodies useful in treatment methods and diagnostic methods. Such antibodies can neutralize the Tat protein in vitro and in vivo, and can be useful in inhibiting HIV infection, by passive protection or inducing an immune response.

Methods for producing an antibody include administering a peptide having at least about 15 to about 21 amino acid residues from the amino terminus region of Tat, optionally conjugated to a viral carrier protein, wherein the amino acid sequence comprises at least amino acid residue 1, 7 and 12 from the amino terminus region of Tat.

The present invention provides for isolated and purified polynucleotides that encode peptides specific for linear epitopes on the amino terminus of Tat. In a preferred embodiment, the nucleotide sequences of the present invention are DNA molecules. Even more preferred, a polynucleotide of the present invention encodes a peptide comprising the amino acid residue sequences of SEQ ID NOs: 1, 2, 3, 4, 5, or 6.

Yet another aspect of the present invention contemplates an isolated and purified polynucleotide comprising a base sequence that is identical or complementary to a segment of at least 10 contiguous bases that encodes for a peptide comprising the amino acid residue sequences of SEQ ID NOs: 1, 2, 3, 4, 5 or 6, wherein the polynucleotide hybridizes to a polynucleotide that encodes a linear epitope peptide of the amino terminus of Tat.

In another embodiment, the present invention contemplates an isolated and purified linear epitope peptide of the amino terminus of Tat that is a recombinant peptide that comprises the amino acid residue sequence of SEQ ID NOs: 1, 2, 3, 4, 5 or 6.

In addition to the peptide of SEQ ID NO. 1, 2, 3, 4, 5 or 6, additional peptide sequence modification are included, such as minor variations, deletions, substitutions or derivitizations of the amino acid sequence of the sequences disclosed herein, so long as the peptide has substantially the same activity or function as the unmodified peptides. A modified peptide will retain activity or function associated with the unmodified peptide, the modified peptide will generally have an amino acid sequence “substantially homologous” with the amino acid sequence of the unmodified sequence.

In an alternative embodiment, the present invention provides an expression vector comprising a polynucleotide that encodes for a Tat amino terminus linear epitope peptide of the present invention. Preferably, an expression vector of the present invention comprises a polynucleotide that encodes a peptide comprising the amino acid residue sequence of SEQ ID NOs: 1, 2, 3, 4, 5 or 6.

In yet another embodiment, the present invention provides a recombinant host cell transfected with a polynucleotide that encodes a Tat amino terminus linear epitope peptide of the present invention. Preferably, a recombinant host cell of the present invention is transfected with a polynucleotide that encodes for a peptide having an amino acid residue sequence selected from SEQ ID NOs: 1, 2, 3, 4, 5 or 6.

In yet another embodiment, the present invention contemplates a process of preparing a Tat amino terminus linear epitope peptide of the present invention comprising transfecting a cell with polynucleotide that encodes the Tat amino terminus linear epitope peptide to produce a transformed host cell and maintaining the transformed host cell under biological conditions sufficient for expression of the peptide. Preferably, the transformed host cell is a eukaryotic cell, such as COS or CHO cell or a prokaryotic cell, such a bacterial cell of Escherichia coli. Even more preferably, a polynucleotide transfected into the transformed cell comprises a nucleotide base sequence that encodes for a peptide having an amino acid residue sequence selected from SEQ ID NOs: 1, 2, 3, 4, 5 or 6.

In still another embodiment, the present invention provides an antibody immunoreactive with a Tat amino terminus linear epitope peptide of the present invention, wherein the antibody can be monoclonal or polyclonal, but preferably monoclonal. Preferably, the Tat amino terminus linear epitope peptide comprises the amino acid residue sequence of SEQ ID NOs: 1, 2, 3, 4, 5 or 6.

In yet another aspect, the present invention relates to antibodies, including polyclonal and monoclonal, and production thereof, wherein the antibody is immunoreactive with a Tat amino terminus linear epitope peptide of the present invention comprising the steps of:
(a) introducing a Tat amino terminus linear epitope peptide of the present invention into a live animal subject; and
(b) recovering the antibody

Another aspect of the present invention relates to a method of expressing a Tat amino terminus linear epitope peptide of the present invention comprising the steps of:

(a) transfecting a recombinant host cell with a polynucleotide that encodes a Tat amino terminus linear epitope peptide of the present invention; (b) culturing the host cell under conditions sufficient for expression of the polypeptide; (c) recovering the polypeptide.

Preferably, the host cell is transfected with the polynucleotide of that encodes for a peptide having an amino acid residue sequence selected from SEQ ID NOs: 1, 2, 3, 4, 5 or 6. Alternatively, steps (a), (b) and (c) can be avoided by use of a synthetic polypeptide.

Alternatively, the present invention provides a process of detecting a Tat peptide, wherein the process comprises immunoreacting the peptide with an antibody prepared according to the process described above to form an antibody-peptide conjugate, and detecting the conjugate.

In another aspect, the present invention contemplates a diagnostic assay kit for detecting the presence of an immunoreactive antibody to a Tat amino terminus linear epitope in a biological sample, where the kit comprises a first container containing at least one Tat amino terminus linear epitope peptide of the present invention capable of immunoreacting with a linear epitope antibody in the biological sample, with the peptide in an amount sufficient to perform at least one assay. Preferably, an assay kit of the invention further comprises a second container containing a second antibody with an indicator that immunoreacts with a binding antibody to the peptide.

In the alternative, antibodies specific for Tat amino terminus linear epitope peptides of the present invention may be used in assays for the detection of HIV-1 tat protein.

Yet another aspect relates to a method of making a Tat amino terminus linear epitope peptide/viral carrier protein chimera comprising the steps of:

covalently attaching an immunogenic viral carrier protein to the Tat amino terminus linear epitope peptide to form the chimera. The Tat amino terminus linear epitope peptide/viral carrier protein chimera may be administered alone or in a pharmaceutical composition as a vaccine in a therapeutically effective amount to elicit an enhanced immune response or a protective immune response in an animal.

Other features and advantages of the invention will be apparent from the following detailed description, drawings and claims.

### BRIEF DESCRIPTION OF THE FIGURES

FIG. 1 shows the variability of the Tat protein sequence. A total of 1,360 sequences derived from the Los Alamos database were used for sequence analysis with the BioEdit biological sequence alignment editor (27). The entropy plot reflects the degree of variability at each position in the full-length Tat protein.

FIGS. 2A and B shows sequences of 36 peptides (SEQ ID NOs. 16-51) used to construct the array. Peptides are organized into six groups, with six peptides in each group. Peptides overlap by 5 aa at both ends. The first three peptides (from the top of each group) are derived from clade B sequences, and the next two are derived from clade C. The sixth peptide in each group is a scrambled (control) peptide.

FIGS. 3A and B show sera from Tat-immunized macaques recognize multiple epitopes in lade B and lade C Tat sequences. The reactivities of individual sera of immunized monkeys with peptides in ELISA are presented according to the optical densities in duplicate measurements. Individual values are color coded (indicated in the top right corner, with red showing the strongest reaction). For each peptide set, optical densities of the wells containing the control (scrambled) peptide were subtracted from the optical densities of the wells containing the test peptides. The subtraction was performed for each animal serum individually. (A) Sera from Tat-immunized macaques; (B) sera from animals immunized with Tat toxoid.

FIGS. 4A and B show a monoclonal antibody to the basic region recognizes a variable epitope. The monoclonal antibody 9A11 recognizes only peptides (top panel) or Tat proteins (middle panel) containing the 57RPPQ60 sequence (present in IIIB and not present in 89.6). The amino terminus antibody TRI recognizes an epitope conserved in both proteins (bottom panel). O.D., optical density. Error bars indicate standard deviations

FIGS. 5A and B show a small fraction of soluble Tat is competent for internalization in Jurkat cells. A solution of 1 μg of Tat per ml in RPMI plus 0.1% ultrapure BSA was mixed with Jurkat cells, and then the cells were removed by centrifugation, the same Tat solution was added to fresh Jurkat cells, and the procedure was repeated for a third serial passage. (A) A Western blot assay showed that the majority of Tat uptake occurred in the first cell exposure (lane 2) and that there was minimal uptake in the second (lane 3) or third (lane 4) passage. The concentration of soluble Tat was only slightly decreased from starting levels (lane 1, before cell addition). (B) To show that Tat was stable in solution, we used either fresh Tat (lane 1) or Tat incubated in medium for 90 min at 37° C. (lane 2) before addition to Jurkat cells.

FIGS. 6A, B and C show antibody neutralization of Tat uptake and transactivation activities. (A) The mouse monoclonal antibodies 9A11 and TR1 blocked Tat uptake into Jurkat cells. A Western blot assay showed a sharp reduction in nuclear and cytoplasmic Tat by 9A11 and TR1 but not by an irrelevant control mouse monoclonal antibody. (B) A similar result was observed with protein G-purified IgG from Tat-immunized macaques. Tat internalization was measured by Western blotting of Jurkat nuclear extracts; we show a typical blot and the data collected with a phosphorimager. Uptake levels were normalized to the value observed in the presence of non immune, control macaque Ig (100% in lane 7). In the absence of IgG (lane 2), uptake was above the control level. Purified IgG from four immunized macaques all blocked Tat uptake and nuclear internalization. Error bars indicate standard deviations. (C) These same monoclonal antibodies and IgG neutralized Tat in the transactivation assay. The data are represented as the optical density (O.D.) from the ELISA for p24 capsid antigen at 96 h after Tat addition. The monoclonal antibodies 9A11 and TR1 had the strongest neutralizing effect and were comparable to IgG for macaque 96079. Macaques 96116, 96122, and 96134 showed a lesser but distinct neutralization of Tat transactivation compared to the non immune IgG control.

FIG. 7 shows Tat transactivation of virus production has a normal dose response in CD4⁺ HeLa cells carrying Tat-defective provirus. Tat was added at 0.3 to 10 μg/ml in RPMI plus 0.1% ultrapure BSA. The incubation and culture were as described in Materials and Methods. At 96 h after Tat addition, culture fluids were tested for virus levels by using a capture ELISA for p24 capsid antigen. Data are represented as optical density (O.D.) in the p24 ELISA for each Tat concentration. Error bars indicate standard deviations.

### DETAILED DESCRIPTION OF THE INVENTION

In order to facilitate review of the various embodiments of the invention and provide an understanding of the various elements and constituents used in making and using the present invention, the following terms used in the invention description have the following meanings.

### DEFINITIONS

A method of treating a viral infection is meant herein to include “prophylactic” treatment or “therapeutic” treatment. A “prophylactic” treatment is a treatment administered to a subject who does not exhibit signs of a disease or who exhibits early signs of the disease for the purpose of decreasing the risk of developing pathology associated with the disease.

The term “therapeutic,” as used herein, means a treatment administered to a subject who exhibits signs of pathology for the purpose of diminishing or eliminating those signs.

The term “therapeutically effective amount,” as used herein means an amount of compound that is sufficient to provide a beneficial effect to the subject to which the compound is administered. A beneficial effect means rendering a virus incompetent for replication, inhibition of viral replication, inhibition of infection of a further host cell, or increasing CD4 T-cell count, for example.

The term “specific binding,” as used herein, in reference to the interaction of an antibody and a protein or peptide, means that the interaction is dependent upon the presence of a particular structure (i.e., the antigenic determinant or epitope) on the protein; in other words, the antibody is recognizing and binding to a specific protein structure rather than to proteins in general.

The term “antibody,” as used herein, means intact molecules as well as fragments thereof, such as Fa, F(ab′)₂, and Fv, which are capable of binding the epitopic determinant.

The terms “peptide,” “polypeptide” and “protein,” as used herein, are used interchangeably to denote a sequence polymer of at least two amino acids covalently linked by an amide bond.

The term “homologous,” as used herein, refers to amino acid sequence similarity between two peptides. When an amino acid position in both of the peptides is occupied by identical amino acids, they are homologous at that position. Thus by “substantially homologous” means an amino acid sequence that is largely, but not entirely, homologous, and which retains most or all of the activity as the sequence to which it is homologous. As used herein, “substantially homologous” as used herein means that a sequence is at least 50% identical, and preferably at least 75% and more preferably 95% homology to the reference peptide.

1. The Invention

The present invention provides DNA segments, purified polypeptides, methods for obtaining antibodies, methods of cloning and using recombinant host cells necessary to obtain and use recombinant Tat amino terminus linear epitope peptides of the present invention, optionally conjugate to a carrier protein. Preferably, the Tat amino terminus linear epitope peptides of the present invention are conjugated to a viral carrier protein. Thus, the present invention is generally concerned with compositions and methods for the preparation and use of Tat amino terminus linear epitope peptides.

II. Polynucleotides

A. Isolated and Purified Polynucleotides That Encode Tat Amino Terminus Linear Epitope Peptides

In one aspect, the present invention provides an isolated and purified polynucleotide that encodes a Tat amino terminus linear epitope peptide. In a preferred embodiment, the polynucleotide of the present invention is a DNA molecule. Even more preferred, a polynucleotide of the present invention is selected from SEQ ID NO. 7-12 and encodes a polypeptide comprising the amino acid residue sequence selected from SEQ ID NOs: 1, 2, 3, 4, 5 or 6.

As used herein, the term “polynucleotide” means a sequence of nucleotides connected by phosphodiester linkages. Polynucleotides are presented herein in the direction from the 5′ to the 3′ direction. A polynucleotide of the present invention can be a deoxyribonucleic acid (DNA) molecule or ribonucleic acid (RNA) molecule. Where a polynucleotide is a DNA molecule, that molecule can be a gene or a cDNA molecule. Nucleotide bases are indicated herein by a single letter code: adenine (A), guanine (G), thymine (T), cytosine (C), inosine (I) and uracil (U).

A polynucleotide of the present invention can be prepared using standard techniques well known to one of skill in the art. The preparation of a cDNA molecule encoding a Tat amino terminus linear epitope peptide of the present invention is described hereinafter.

The present invention provides an isolated and purified polynucleotide that encodes a Tat amino terminus linear epitope peptide, where the polynucleotide is prepared by a process comprising the steps of constructing a library of cDNA clones from a cell that expresses the polypeptide; screening the library with a labeled cDNA probe prepared from RNA that encodes the polypeptide; and selecting a clone that hybridizes to the probe. Preferably, the polynucleotide of the invention encodes a polypeptide that has the amino acid residue sequence of SEQ ID NOs: 1, 2, 3, 4, 5 or 6.

B. Probes and Primers

In another aspect, DNA sequence information provided by the present invention allows for the preparation of relatively short DNA (or RNA) sequences having the ability to specifically hybridize to gene sequences of the selected polynucleotide disclosed herein. In these aspects, nucleic acid probes of an appropriate length are prepared based on a consideration of a selected nucleotide sequence. The ability of such nucleic acid probes to specifically hybridize to a polynucleotide encoding a Tat amino terminus linear epitope peptide lends them particular utility in a variety of embodiments. Most importantly, the probes can be used in a variety of assays for detecting the presence of complementary sequences in a given sample.

In certain embodiments, it is advantageous to use oligonucleotide primers. The sequence of such primers is designed using a polynucleotide of the present invention for use in detecting, amplifying or mutating a defined segment of a gene or polynucleotide that encodes a Tat amino terminus linear epitope peptide of the present invention from mammalian cells using PCR technology.

To provide certain of the advantages in accordance with the present invention, a preferred nucleic acid sequence employed for hybridization studies or assays includes probe molecules that are complementary to at least a 5 to 15 nucleotide stretch of a polynucleotide that encodes a Tat amino terminus linear epitope peptide. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. Such fragments can be readily prepared by, for example, directly synthesizing the fragment by chemical means, by application of nucleic acid reproduction technology, such as the PCR or by excising selected DNA fragments from recombinant plasmids containing appropriate inserts and suitable restriction enzyme sites.

Accordingly, a polynucleotide probe molecule of the invention can be used for its ability to selectively form duplex molecules with complementary stretches of the gene. Depending on the application envisioned, one will desire to employ varying conditions of hybridization to achieve varying degree of selectivity of the probe toward the target sequence. For applications requiring a high degree of selectivity, one will typically desire to employ relatively stringent conditions to form the hybrids. For example, one will select relatively low salt and/or high temperature conditions, such as provided by 0.02M-0.15M NaCl at temperatures of 50° C. to 70° C. Those conditions are particularly selective, and tolerate little, if any, mismatch between the probe and the template or target strand.

Of course, for some applications, for example, where one desires to prepare mutants employing a mutant primer strand hybridized to an underlying template or where one seeks to isolate a peptide coding sequence from other cells, functional equivalents, or the like, less stringent hybridization conditions are typically needed to allow formation of the heteroduplex. In these circumstances, one can desire to employ conditions such as 0.15M-0.9M salt, at temperatures ranging from 20° C. to 70° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

In general, it is envisioned that a hybridization probe described herein is useful both as a reagent in solution hybridization as well as in embodiments employing a solid phase. In embodiments involving a solid phase, the test DNA (or RNA) is adsorbed or otherwise affixed to a selected matrix or surface. This fixed nucleic acid is then subjected to specific hybridization with selected probes under desired conditions. The selected conditions depend as is well known in the art on the particular circumstances and criteria required (e.g., on the G+C content, type of target nucleic acid, source of nucleic acid, size of hybridization probe). Following washing of the matrix to remove non specifically bound probe molecules, specific hybridization is detected, or even quantified, by means of the label.

III. A Tat Amino Terminus Linear Epitope Peptide

In one embodiment, the present invention contemplates an isolated and purified Tat amino terminus linear epitope peptide. Preferably, the Tat amino terminus linear epitope peptide of the present invention comprises the amino acid residue sequence of SEQ ID NOs: 1, 2, 3, 4, 5, or 6.

Polypeptides are disclosed herein as amino acid residue sequences. Those sequences are written left to right in the direction from the amino to the carboxyl terminus. In accordance with standard nomenclature, amino acid residue sequences are denominated by either a single letter or a three letter code as indicated below.

| Amino Acid Residue | 3-Letter Code | 1-Letter Code |
|---|---|---|
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Modifications and changes can be made in the structure of a polypeptide of the present invention and still obtain a molecule having Tat amino terminus linear epitope peptide like characteristics. For example, certain amino acids can be substituted for other amino acids in a sequence without appreciable loss of peptide activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art (Kyte, J. and R. F. Doolittle 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within .±0.2 is preferred, those that are within .±0.1 are particularly preferred, and those within .±0.0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e. with a biological property of the polypeptide. As detailed in U.S. Pat. No. 4,554,101, the following hydrophilicity values have been assigned to amino acid residues: arginine (+3.0); lysine (+3.0); aspartate (+3.0.±0.1); glutamate (+3.0.±0.1); serine (+0.3); asparagine (+0.2); glutamine (+0.2); glycine (0); proline (−0.5.±0.1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within .±0.2 is preferred, those that are within .±0.1 are particularly preferred, and those within .±0.0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 1, below). The present invention thus contemplates functional or biological equivalents of a peptide as set forth above.

TABLE 1

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

Biological or functional equivalents of a polypeptide can also be prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. As noted above, such changes can be desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed.

In general, the technique of site-specific mutagenesis is well known in the art, as exemplified by Adelman et al., (1983). As will be appreciated, the technique typically employs a phage vector, which can exist in both a single stranded and double stranded form. Typical vectors useful in site-directed mutagenesis include vectors such as the M13 phage (Messing et al., 1981). These phage are commercially available and those of skill in the art generally know their use.

In general, site-directed mutagenesis in accordance herewith is performed by first obtaining a single-stranded vector which includes within its sequence a DNA sequence which encodes all or a portion of the Tat amino terminus linear epitope peptide sequence selected. An oligonucleotide primer bearing the desired mutated sequence is prepared and annealed to the singled-stranded vector, and extended by the use of enzymes such as E. coli polymerase I Klenow fragment, in order to complete the synthesis of the mutation bearing strand. Thus, a heteroduplex is formed wherein one strand encodes the original non mutated sequence and the second strand bears the desired mutation. This heteroduplex vector is then used to transform appropriate cells such as E. coli cells and clones are selected which include recombinant vectors bearing the mutation. Commercially available kits come with all the reagents necessary, except the oligonucleotide primers.

A polypeptide of the present invention is prepared by standard techniques well known to those skilled in the art. Such techniques include, but are not limited to, isolation and purification from tissues known to contain that polypeptide, expression from cloned DNA that encodes such a polypeptide using transformed cells or use of synthetic peptide production systems.

IV. Expression Vectors

The present invention provides expression vectors comprising polynucleotide that encode Tat amino terminus linear epitope peptides of the present invention. Preferably, expression vectors of the present invention comprise polynucleotides that encode polypeptides comprising the amino acid residue sequence of SEQ ID NOs: 1, 2, 3, 4, 5 or 6. More preferably, expression vectors of the invention comprise polynucleotides linked to a nucleotide sequence that expresses a carrier protein, such as a viral carrier protein.

The nucleotide sequences may be operatively linked to an enhancer-promoter. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promote” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter.

Preferably, expression vectors of the present invention comprise polynucleotides that encode polypeptides comprising the amino acid residue sequence of SEQ ID NOs:1, 2, 3, 4, 5 or 6. An expression vector can include a Tat amino terminus linear epitope peptide coding region itself or coding regions bearing selected alterations or modifications in the basic coding region of such a Tat amino terminus linear epitope peptide. Alternatively, such vectors or fragments can code larger polypeptides or peptides that nevertheless include the basic coding region in multiple positions in the vector.

Exemplary vectors include the mammalian expression vectors of the pCMV family including pCMV6b and pCMV6c (Chiron Corp., Emeryville Calif.) and pRc/CMV (Invitrogen, San Diego, Calif.). In certain cases, and specifically in the case of these individual mammalian expression vectors, the resulting constructs can require co-transfection with a vector containing a selectable marker such as pSV2neo. Via co-transfection into a dihydrofolate reductase-deficient Chinese hamster ovary cell line, such as DG44, clones expressing the peptides of the present invention by virtue of DNA incorporated into such expression vectors can be detected.

A DNA molecule of the present invention can be incorporated into a vector using a number of techniques that are well known in the art. For instance, the vector pUC18 has been demonstrated to be of particular value. Likewise, the related vectors M13mp18 and M13mp19 can be used in certain embodiments of the invention, in particular, in performing dideoxy sequencing.

An expression vector of the present invention is useful both as a means for preparing quantities of a Tat amino terminus linear epitope peptide-encoding DNA itself, and as a means for preparing the encoded peptides. It is contemplated that where Tat amino terminus linear epitope peptides of the invention are made by recombinant means, one can employ either prokaryotic or eukaryotic expression vectors as shuttle systems. Such a system is described herein which allows the use of bacterial host cells as well as eukaryotic host cells.

Where expression of recombinant polypeptide of the present invention is desired and a eukaryotic host is contemplated, it is most desirable to employ a vector, such as a plasmid, that incorporates a eukaryotic origin of replication. Additionally, for the purposes of expression in eukaryotic systems, one desires to position the Tat amino terminus linear epitope peptide encoding sequence adjacent to and under the control of an effective eukaryotic promoter. To bring a coding sequence under control of a promoter, whether it is eukaryotic or prokaryotic, what is generally needed is to position the 5′ end of the translation initiation side of the proper translational reading frame of the polypeptide between about 1 and about 50 nucleotides 3′ of or downstream with respect to the promoter chosen. Furthermore, where eukaryotic expression is anticipated, one would typically desire to incorporate into the transcriptional unit, which includes the Tat amino terminus linear epitope peptide, an appropriate polyadenylation site.

The pRc/CMV vector (available from Invitrogen) is an exemplary vector for expressing a Tat amino terminus linear epitope peptide in mammalian cells, particularly COS and CHO cells. A polypeptide of the present invention under the control of a CMV promoter can be efficiently expressed in mammalian cells. The pCMV plasmids are a series of mammalian expression vectors of particular utility in the present invention. The vectors are designed for use in essentially all cultured cells and work extremely well in SV40-transformed simian COS cell lines. The pCMV1, 2, 3, and 5 vectors differ from each other in certain unique restriction sites in the polylinker region of each plasmid. The pCMV4 vector differs from these 4 plasmids in containing a translation enhancer in the sequence prior to the polylinker. While they are not directly derived from the pCMV1-5 series of vectors, the functionally similar pCMV6b and c vectors are available from the Chiron Corp. of Emeryville, Calif. and are identical except for the orientation of the polylinker region which is reversed in one relative to the other. The pCMV vectors have been successfully expressed in simian COS cells, mouse L cells, CHO cells, and HeLa cells.

V. Transfected Cells

In yet another embodiment, the present invention provides recombinant host cells transformed or transfected with a polynucleotide that encodes a Tat amino terminus linear epitope peptide. Preferably, recombinant host cells of the present invention are transfected with a polynucleotide that encodes for a peptide having an amino acid residue sequence selected from SEQ ID NOs 1, 2, 3, 4, 5 or 6. Means of transforming or transfecting cells with exogenous polynucleotide such as DNA molecules are well known in the art and include techniques such as calcium-phosphate- or DEAE-dextran mediated transfection, protoplast fusion, electroporation, liposome mediated transfection, direct microinjection and adenovirus infection.

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

In another aspect, the recombinant host cells of the present invention are prokaryotic host cells. Preferably, the recombinant host cells of the invention are bacterial cells of Escherichia coli. In general, prokaryotes are preferred for the initial cloning of DNA sequences and constructing the vectors useful in the present invention. For example, E. coli K12 strains can be particularly useful. Other microbial strains that can be used include E. coli B, and E. coli X1776 (ATCC No. 31537). These examples are, of course, intended to be illustrative rather than limiting.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli can be transformed using pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own polypeptides.

Those promoters most commonly used in recombinant DNA construction include the beta.-lactamase (penicillinase) and lactose promoter systems and a tryptophan (TRP) promoter system. While these are the most commonly used, other microbial promoters have been discovered and utilized, and details concerning their nucleotide sequences have been published, enabling a skilled worker to introduce functional promoters into plasmid vectors.

In addition to microorganisms, cultures of cells derived from multicellular organisms can also be used as hosts. In principle, any such cell culture is workable, whether from vertebrate or invertebrate culture. However, interest has been greatest in vertebrate cells, and propagation of vertebrate cells in culture (tissue culture) has become a routine procedure in recent years. Examples of such useful host cell lines are AtT-20, VERO and HeLa cells, Chinese hamster ovary e (CHO) cell lines, and W138, BHK, COSM6, COS-1, COS-7, 293 and MDCK cell lines. Expression vectors for such cells ordinarily include (if necessary) an origin of replication, a promoter located upstream of the gene to be expressed, along with any necessary ribosome binding sites, RNA splice sites, polyadenylation site, and transcriptional terminator sequences.

VI. Preparing A Tat Amino Terminus Linear Epitope Peptide

Regulatory Polypeptide

In yet another embodiment, the present invention contemplates a process of preparing a Tat amino terminus linear epitope peptide comprising transfecting cells with a polynucleotide that encodes a Tat amino terminus linear epitope peptide to produce transformed host cells; and maintaining the transformed host cells under biological conditions sufficient for expression of the polypeptide. As stated above, the transformed host cells may be eukaryotic cells are prokaryotic cells. More preferably, the polynucleotide transfected into the transformed cells comprise the nucleotide base sequence that encodes for a peptide having an amino acid residue sequence of SEQ ID NOs: 1, 2, 3, 4, 5 or 6.

Following transfection, the cell is maintained under culture conditions for a period of time sufficient for expression of the Tat amino terminus linear epitope peptide. Culture conditions are well known in the art and include ionic composition and concentration, temperature, pH and the like. Typically, transfected cells are maintained under culture conditions in a culture medium. Suitable medium for various cell types are well known in the art. In a preferred embodiment, temperature is from about 20° C. to about 50° C. pH is preferably from about a value of 6.0 to a value of about 8.0, more preferably from about a value of about 6.8 to a value of about 7.8 and, most preferably about 7.4. Other biological conditions needed for transfection and expression of an encoded protein are well known in the art.

Transfected cells are maintained for a period of time sufficient for expression of the Tat amino terminus linear epitope peptide. A suitable time depends inter alia upon the cell type used and is readily determinable by a skilled artisan. Typically, maintenance time is from about 2 to about 14 days.

The recombinant Tat amino terminus linear epitope peptide is recovered or collected either from the transfected cells or the medium in which those cells are cultured. Recovery comprises isolating and purifying the recombinant polypeptide. Isolation and purification techniques for polypeptides are well known in the art and include such procedures as precipitation, filtration, chromatography, electrophoresis and the like.

VII. Antibodies

In still another embodiment, the present invention provides antibodies immunoreactive with Tat amino terminus linear epitope peptides of the present invention. Preferably, the antibodies of the invention are monoclonal antibodies. More preferably, the Tat amino terminus linear epitope peptides comprise the amino acid residue sequence of SEQ ID NOs: 1, 2, 3, 4, 5 or 6. Means for preparing and characterizing antibodies are well known in the art.

Briefly, a polyclonal antibody is prepared by immunizing an animal with an immunogen comprising a polypeptide or polynucleotide of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given polypeptide or polynucleotide may vary in its immunogenicity. It is often necessary therefore to couple the immunogen (e.g., a polypeptide or polynucleotide) of the present invention) with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide or a polynucleotide to a carrier protein are well known in the art and include glutaraldehyde, M maleimidobenzoyl-n-hydroxysuccinimide ester, carbodiimide and bis-biazotized benzidine.

As is also well known in the art, immunogenicity to a particular immunogen can be enhanced by the use of non specific stimulators of the immune response known as adjuvants. Exemplary and preferred adjuvants include complete Freund's adjuvant, incomplete Freund's adjuvants and aluminum hydroxide adjuvant.

The amount of immunogen used of the production of polyclonal antibodies varies inter alia, upon the nature of the immunogen as well as the animal used for immunization. A variety of routes can be used to administer the immunogen (subcutaneous, intramuscular, intradermal, intravenous and intraperitoneal. The production of polyclonal antibodies is monitored by sampling blood of the immunized animal at various points following immunization. When a desired level of immunogenicity is obtained, the immunized animal can be bled and the serum isolated and stored.

Typically, a monoclonal antibody of the present invention can be readily prepared by a technique which involves first immunizing a suitable animal with a selected antigen (e.g., a polypeptide or polynucleotide of the present invention) in a manner sufficient to provide an immune response. Rodents such as mice and rats may be used. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non fused parental cells, for example, by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine. This culturing provides a population of hybridomas from which specific hybridomas are selected. Typically, selection of hybridomas is performed by culturing the cells by single-clone dilution in microliter plates, followed by testing the individual clonal supernatants for reactivity with an antigen polypeptide. The selected clones can then be propagated indefinitely to provide the monoclonal antibody.

By way of specific example, to produce an antibody of the present invention, mice or rabbits are injected intraperitoneally with between about 1-200 ug of an antigen comprising a polypeptide of the present invention. B lymphocyte cells are stimulated to grow by injecting the antigen in association with an adjuvant such as complete Freund's adjuvant. At some time (e.g., at least two weeks) after the first injection, the mice or rabbits are boosted by injection with a second dose of the antigen mixed with incomplete Freund's adjuvant. A few weeks after the second injection, mice are tail bled and the sera titered by immunoprecipitation against radiolabeled antigen. Preferably, the process of boosting and titering is repeated until a suitable titer is achieved. The spleen of the mouse or rabbit with the highest titer is removed and the spleen lymphocytes are obtained by homogenizing the spleen with a syringe. Typically, a spleen from an immunized animal contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like myeloma cells, hybridoma cells grow indefinitely in culture. Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media. Each of the surviving hybridoma cells produces a single antibody. These cells are then screened for the production of the specific antibody immunoreactive with an antigen/polypeptide of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific polypeptides of the invention can be recognized as antigens, and thus identified. Once identified, those polypeptides can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide is then easily removed from the substrate and purified.

VIII. Pharmaceutical Compositions

In a preferred embodiment, the present invention provides pharmaceutical compositions comprising at least one Tat amino terminus linear epitope peptide of the present invention, preferably, conjugated to a viral carrier protein and a physiologically acceptable carrier. More preferably, a pharmaceutical composition comprises a Tat amino terminus linear epitope peptide having the amino acid residue sequence of SEQ ID NO: 1, 2, 3, 4, 5, and/or 6.

In the alternative, the pharmaceutical composition of the invention may comprise a polynucleotide that encodes a Tat amino terminus linear epitope peptide of the present invention, optionally linked to a nucleotide sequence encoding a viral protein and a physiologically acceptable carrier.

The viral carrier protein may include, but is not limited to the gag, env, nef proteins or fragments thereof of HIV.

A composition of the present invention is typically administered parenterally in dosage unit formulations containing standard, well-known nontoxic physiologically acceptable carriers, adjuvants, and vehicles as desired. The term parenteral as used herein includes intravenous, intramuscular, intraarterial injection, or infusion techniques.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

The compositions of the invention are administered in substantially non toxic dosage concentrations sufficient to ensure the release of a sufficient dosage unit of the present peptides into the patient to provide the desired inhibition of Tat into the T cells. The actual dosage administered will be determined by physical and physiological factors such as age, body weight, severity of condition, and/or clinical history of the patient.

Further, the therapeutic compositions according to the present invention may be employed in combination with other-therapeutic agents for the treatment of viral infections or conditions. Examples of such additional therapeutic agents include agents that are effective for the treatment of viral infections or associated conditions such as immunomodulatory agents such as thymosin, ribonucleotide reductase inhibitors such as 2-acetylpyridine 5-[(2-chloroanilino) thiocarbonyl) thiocarbonohydrazone, interferons such as alpha-interferon, 1-beta-D-arabinofuranosyl-5-(1-propynyl)uracil, 3′-azido-3′-deoxythymidine, ribavirin and phosphonoformic acid.

IX. A Process of Detecting Polypeptides

The present invention provides a process of detecting a Tat amino terminus linear epitope peptide, wherein the process comprises immunoreacting the Tat amino terminus linear epitope peptide with antibodies prepared according to a process described above to form an antibody-polypeptide conjugate and detecting the conjugates.

X. Screening Assays

In yet another aspect, the present invention contemplates a process of screening for cross-reactive antibodies that interact with different clades of HIV, the process comprising the steps of providing a Tat amino terminus linear epitope peptide of the present invention and testing the ability of antisera to interact with that peptide.

Screening assays of the present invention generally involve determining the ability of antibodies in antisera to bind to the peptide. The peptides of the present invention can be coupled to a solid support. The solid support can be agarose beads, polyacrylamide beads, polyacrylic beads or other solid matrices capable of being coupled to proteins. Well known coupling agents include cyanogen bromide, carbonyidiimidazole, tosyl chloride, and glutaraldebyde.

In a typical screening assay for identifying antibodies in antisera for Tat amino terminus linear epitope peptides, one employs an amount of a peptide in an appropriate assay buffer at an appropriate pH. The antisera is added to the admixture in convenient concentrations and any interaction between antibodies and the peptide is monitored.

Accordingly, it is proposed that this aspect of the present invention provides those of skill in the art with methodology to measure the proportion of serum antibodies that bind Tat amino terminus linear epitope peptides. Antisera is mixed with Tat amino terminus linear epitope peptides of the present invention and is then transferred to an ELISA with Tat protein. If the antisera contain antibodies that bind linear epitopes, the preincubation with the Tat amino terminus linear epitope peptides of the present invention will reduce binding to the Tat protein.

In the alternative, antibodies specific for Tat amino terminus linear epitope peptides of the present invention may be used in assays for the detection of HIV-1 tat protein.

XI. Assay Kits

In another aspect, the present invention contemplates diagnostic assay kits for detecting the presence of antibodies specific for Tat amino terminus linear epitope peptides of the present invention in biological samples, where the kits comprise a first container containing at least one Tat amino terminus linear epitope peptide capable of immunoreacting with antibodies in biological samples, with the in an amount sufficient to perform at least one assay. Preferably, assay kits of the invention further comprise a second container containing a second antibody that immunoreacts with the first antibody. Preferably the antibodies used in the assay kits of the present invention are monoclonal antibodies. Even more preferably, the peptides are affixed to a F solid support. More preferably still, the first and second antibodies comprise an indicator, and, preferably, the indicator is a radioactive label or an enzyme. The reagents of the kit can be provided as a liquid solution, attached to a solid support or as a dried powder. Preferably, when the reagent is provided in a liquid solution, the liquid solution is an aqueous solution. Preferably, when the reagent provided is attached to a solid support, the solid support can be chromatograph media or a microscope slide. When the reagent provided is a dry powder, the powder can be reconstituted by the addition of a suitable solvent. The solvent can be provided.

### EXAMPLES

The main goal of the following testing was to compare antisera from animals immunized with Tat or chemically modified Tat toxoid, in order to define common epitopes that might account for disease attenuation in vaccinated animals. In addition, we characterized the mechanism for Tat neutralization and studied the breadth of the antibody responses to Tat sequences from lade B and lade C viruses.

Materials and Methods

Polyclonal antisera were obtained from healthy rhesus macaques that had been immunized with Tat toxoid or Tat as described previously (37). Briefly, animals were immunized three times by intramuscular injection with polyphosphazene adjuvant (Adjumer) and twice by intramuscular injection of protein in incomplete Freund's adjuvant. Antigen doses ranged from 10 to 60 μg. Sera were collected 8 to 12 days after the last immunization and stored at −130° C. until used.

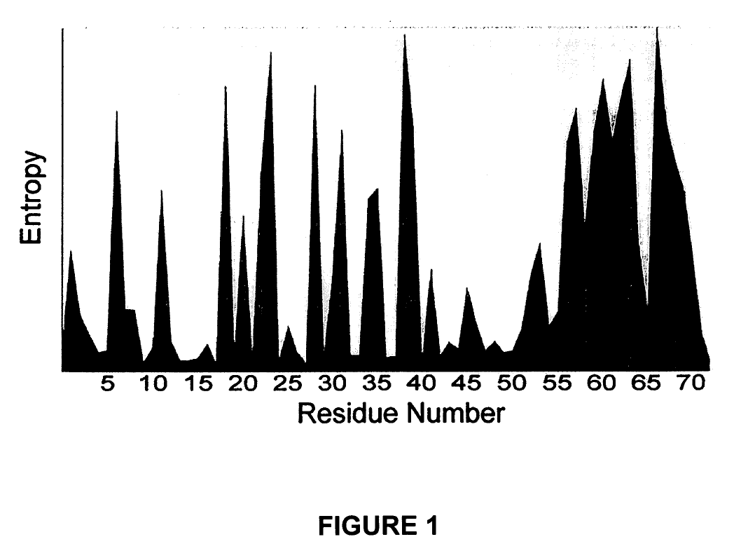

Tat sequence analysis. One thousand three hundred sixty Tat first-exon sequences were obtained from the Los Alamos database. The sequences included roughly 50% clade B, 19% lade C, 13% clade A, 4% lade O, 2% lade D, and 1% lade G. The remainder included clades J, K, M, H, and F and 11% of sequences that were unassigned. Only the first-exon sequences were used to avoid problems with the variable lengths of Tat proteins. The sequences were aligned and analyzed by using the BioEdit biological sequence alignment editor (27) as shown in FIG. 1.

Aligned sequences were compared by using an entropy plot that reflects the amount of variability through each column in the alignment. For entropy plotting, the sequences were treated as a matrix of characters. Entropy in a column position is independent of the total information possible at a given position and depends only upon the frequencies of characters that appear in that column. Entropy was then calculated which gave a measure of uncertainty at each position relative to other positions (27).

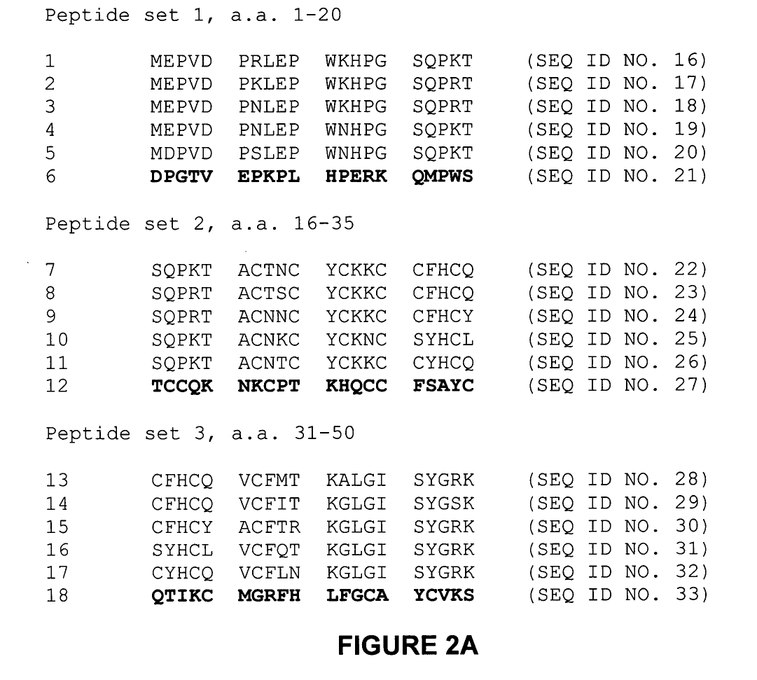

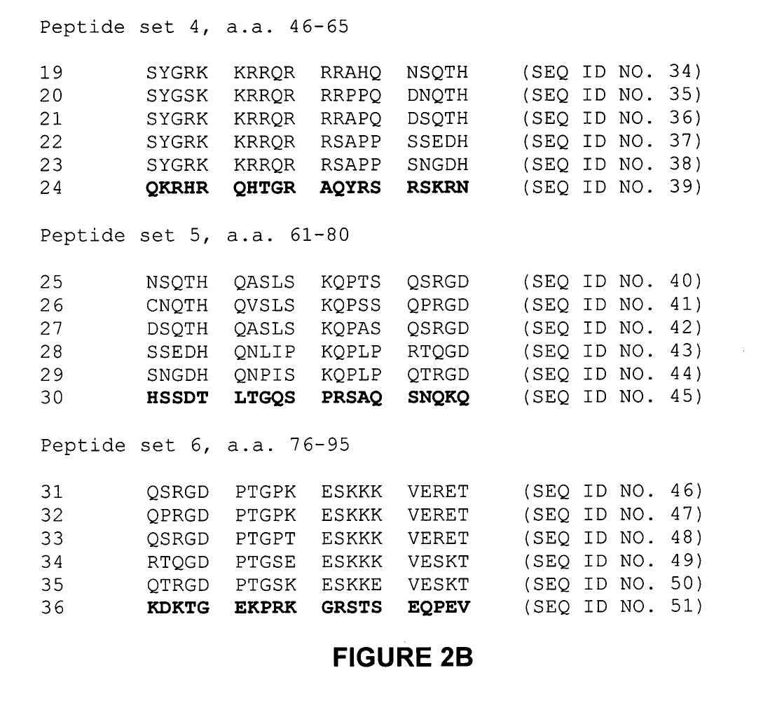

Peptide array. Three representative clade B Tat sequences, a clade C consensus sequence, and an authentic clade C sequence (B.-.NL43E9, B.AU.MBCD36, B.US.SF2, C.BW.96BW17, and Consensus C [Los Alamos HIV database]) were selected for detecting serum antibody responses. For each complete (101- or 102-aa) sequence, synthetic peptides were obtained covering the entire protein. The peptides were 20 aa long and overlapped by 5 aa at the amino terminus and 5 aa at the carboxyl terminus. In addition, a set of scrambled peptide controls was designed. The scrambled peptides had the same amino acid composition as a consensus clade B sequence but were randomized such that no sequence of 3 aa or longer in the scrambled peptide matched any of the other five corresponding peptide sequences. Peptides were synthesized by using 9-fluorenylmethoxy carbonyl chemistry with HATU/DIEA activation at the Biopolymer Core Facility, Department of Microbiology and Immunology, University of Maryland School of Medicine. All peptides were purified by high-pressure liquid chromatography. The integrity of each preparation was confirmed by electrospray ionization mass spectrometry, and all were at least 80% the correct peptide sequence. The peptides were grouped according to their position in the protein sequence and the five Tat peptides and one scrambled peptide were aligned to generate the array used for serology studies, as shown in FIG. 2.

ELISA. The peptides were dissolved in water at 4 mg/ml and stored at −20° C. until used; all peptides were soluble at this concentration. Peptides were adsorbed to enzyme-linked immunosorbent assay (ELISA) plates (Costar, Cambridge, Mass.) by overnight incubation at 4° C. in 100 mM carbonate buffer (pH 9.5) at a concentration of 10 μg per ml. The plates were subsequently washed and treated with 50 mM Tris HCl (pH 7.8)-0.15 M NaCl-0.1% Tween 20. Serum samples were diluted 1:100 in a buffer containing 50 mM Tris HCl (pH 7.8), 0.15 M NaCl, 0.05% Tween 20, 0.5% Triton X-100, and 1% bovine serum albumin (BSA) (Sigma, St. Louis, Mo.). Wells were filled with samples (in duplicate) and incubated for 2 h at room temperature with shaking. After four washes with 50 mM Tris HCl (pH 7.8)-0.15 M NaCl-0.05% Tween 20, an anti-monkey immunoglobulin (IgG) alkaline phosphatase conjugate (affinity purified; Sigma) diluted 1:10,000 in 50 mM Tris HCl (pH 7.8)-0.15 M NaCl-0.05% Tween 20-1% BSA was added and left for 1 h at room temperature. After four washes, the substrate solution was added and plates were incubated at 37° C. for 30 min.

As a control for nonspecific binding, the optical density for each well was measured and subtracted the value for scrambled peptide. Sera from immunized animals (1:100 dilution) did not react significantly with any of the scrambled peptides, indicating that the assay conditions were suitable for detecting specific binding to Tat peptides. Preimmune sera (1:100 dilution) did not react with the array.

Generation of monoclonal antibodies to the Tat protein. BALB/c mice were injected twice with 50 pg of Tat toxoid, first with complete and then with incomplete Freund's adjuvant. Four weeks later, mice were boosted and polyethylene glycol 4000-mediated fusion was performed as described previously (19). Hybridomas were screened by ELISA, positive clones were retested by Western blotting and then cloned, and the monoclonal IgG was purified by protein G affinity chromatography. Hybridoma TR1 reacts with an epitope in aa 1 to 15. Hybridoma 9A11 reacts with the sequence from aa 46 to 60 of the HIV Tat protein sequence (46SYGSKKRRQRRRPPQ60) (SEQ ID NO. 13) that is found in the B.US.YU2, B.AU.MBCD36, and B.US.JRCSF sequences (Los Alamos database).

Expression of glutathione S-transferase-Tat 89.6 fusion protein. aa 1 to 86 of simian/HIV 89.6 Tat were expressed as a glutathione S-transferase fusion protein from the pGEX-5x-2 vector (Amersham Pharmacia, Piscataway, N.J.). The fusion protein was purified using Glutathione Sepharose 4B (Amersham Pharmacia). The purity of the preparation was confirmed by polyacrylamide gel electrophoresis and by Western blotting with monoclonal antibody TR1, which recognizes the amino-terminal peptide of Tat.

Tat internalization assay. Jurkat cells (5×10⁶ per ml) were incubated with recombinant Tat (86 aa) (Advanced Biosciences Laboratories, Kensington, Md.) at 1 μg/ml in RPMI supplemented with 0.1% ultrapure BSA (Panvera, Madison, Wis.) for 2 h at 37° C. The entire culture fluid was removed, a small fraction was saved to measure soluble Tat levels, and the fluid was transferred to fresh Jurkat cells for a second 20-min incubation. The procedure was repeated for a third serial overlay. In some cases Tat was preincubated with monoclonal antibody 9A11 or TR1 or IgG controls at the ratio of 1 μg of Tat to 50 μg of IgG (for a molar ratio of approximately 1:5). After incubation, the cells were washed three times, and then cytoplasmic and nuclear extracts were prepared with the NE-PER kit (Pierce, Rockford, Ill.). Protein concentrations were determined with Coomassie Plus Protein Assay Reagent (Pierce), and Tat was detected in lysates by Western blotting. Briefly, 12.5 μg of each nuclear extract and 50 μg of each cytoplasmic extract were separated by protein gel electrophoresis and transferred to a polyvinylidene difluoride membrane. TR1 monoclonal antibody and subsequently goat anti-mouse alkaline phosphatase conjugate (Sigma) were used to detect Tat on the blots. Membranes were incubated with the enhanced chemiluminescence substrate Lumi-Phos WB (Pierce) and exposed to CL-Xposure film (Pierce). Three nanograms of Tat was used as a positive control.

This assay was used to test whether Tat-specific monoclonal or polyclonal antibodies could block Tat internalization. Tat was preincubated with protein G-purified monkey IgG, purified control IgG, or monoclonal antibodies for 30 min at room temperature. The mixtures were added to Jurkat cells, and the Western blot assay for nuclear accumulation of Tat was done as described above. All samples were within 8% as judged by the band intensity of an internal loading control.

Tat transactivation assay. A CD4⁺ HeLa cell line (kindly provided by Barbara Felber and George Pavlakis) containing an HIV-1 provirus was used that does not express Tat and does not release virus particles (44). Cells were seeded into a 96-well plate at 40,000 cells per well and incubated overnight. The attached cells were washed three times with warmed serum-free RPMI and then overlaid with RPMI-0.1% ultrapure BSA (Panvera) containing Tat protein for 90 min. The Tat solutions were removed and replaced with complete Dulbecco's modified Eagle medium. Culture fluids were collected 96 h later, and cell-free virus was detected with a commercial antigen capture ELISA for p24 capsid antigen (R&D Systems). Data are expressed as the mean optical density±the standard deviation from quadruplicate samples.

This assay was used to measure the ability of monoclonal antibodies or polyclonal antibodies to neutralize the Tat activity. Tat was preincubated with protein G-purified monkey IgG, purified control IgG, or monoclonal antibodies for 30 min at room temperature. The Tat-antibody solution was added to indicator cells, left for 90 min, and then removed and replaced with Dulbecco's modified Eagle medium containing 10% heat-inactivated fetal bovine serum.

Amino acid sequence variation in Tat from different viral isolates. The Tat protein has often been described as having a conserved sequence (20,21). This work and work of others (11,23) support a different interpretation. Analysis of the first exon sequences (aa 1 to 72) from 1,360 Tat sequences in the Los Alamos database revealed significant variability among Tat amino acid sequences as shown in FIG. 1, although there were some islands of sequence conservation. The basic region, consisting of the sequence SYGSKKRRQRRR (SEQ ID NO. 14) for aa 45 to 56, is generally conserved, as are all seven cysteine residues that are required for transactivation activity (30) and pathogenic effects (9). In addition, the amino-terminal sequence is relatively conserved. Other regions of Tat showed substantial variation in this entropy plot.

A peptide microarray for detecting Tat antibodies. Overlapping peptides were synthesized to match three authentic clade B sequences, a lade C consensus sequence, an authentic lade C sequence, and a scrambled peptide control as described in Materials and Methods. Sequences were selected to represent the most common Tat variants. This peptide microarray was tested with Tat-specific monoclonal antibodies TR1 and 9A11 (described below) and with hyperimmune mouse serum raised by immunization with Tat toxoid.

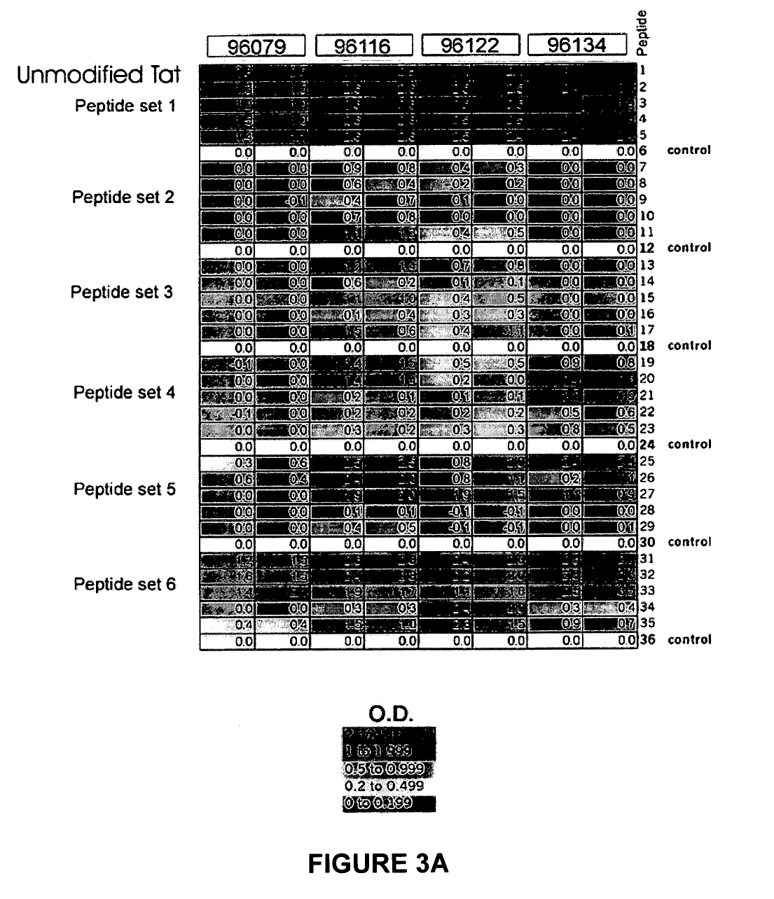

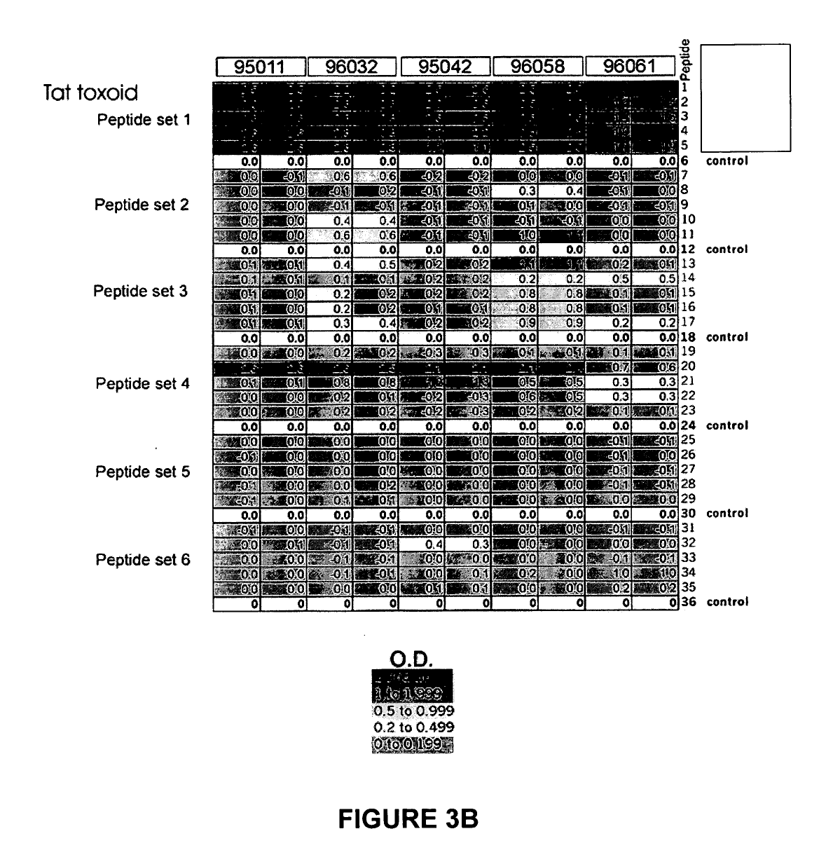

Spectrum of antibodies induced by immunization with unmodified Tat protein. Macaques immunized with Tat developed robust serum antibody responses, with endpoint titers ranging from 1:5,000 to 1:64,000 in ELISA (36). Sera from all immunized macaques reacted strongly with amino terminal peptides (set 1) and did not discriminate B and C lade sequences as shown in FIG. 3. Although there is some variation in the amino-terminal sequences for Tat proteins, there are sufficient conserved residues (including the amino-terminal sequence MEPVD, the sequence LEPW at aa 8 to 11, and the sequence HPGSQP at aa 13 to 18) to account for the cross-reactions. Two of four animals reacted with peptides from sets 2 and 3, and two of four macaques reacted with peptides from set 4, although not all sera reacted with all sequences, indicating the recognition of nonconserved epitopes. All Tat-immunized animals recognized peptides in set 5 as shown in FIG. 3A, although the reaction was restricted to clade B sequences, indicating the presence of a clade-specific epitope. Recognition of the C-terminal portion (aa 76 to 95) was strong in all animals and was not restricted to either lade.

Overall, epitopes in peptide sets 1 (aa 1 to 20) and 6 (aa 76 to 95) were recognized by sera from all animals immunized with Tat. The epitopes appear to be conserved, because there were no consistent differences among individual sequences or across clades. Peptide sets 2 and 3 were recognized by half of the sera. Recognition of peptide set 4 was inconsistent, suggesting the presence of a nonconserved epitope, and recognition of peptide set 5 showed clear evidence for clade B-specific recognition by sera raised against the clade B Tat protein.

Spectrum of antibodies induced by Tat toxoid immunization. Animals immunized with Tat toxoid also developed strong Tat-binding antibodies, with titers similar to those in the group immunized with Tat. However, Tat toxoid elicited a more restricted antibody response, suggesting that carboxymethylation reduced the immunogenicity for some but not all Tat epitopes as shown in FIG. 3B.

Sera from all Tat toxoid-immunized macaques recognized conserved epitopes in the amino-terminal sequence. There was weak recognition of peptides in sets 2 and 3. Peptide 20 from set 4 was recognized strongly by all animals immunized with Tat toxoid, but the epitope appeared to be highly variable and was present only in peptide 20 and not in other peptides from the same set. The epitope in peptide 20 (aa 46 to 65) was mapped by using additional synthetic peptides. Sera from some of the immunized animals preferentially reacted with peptides consisting of aa 46 to 65 and 46 to 60 as shown below in Table 2 below.

TABLE 2

| | Reaction (optical density)^{a} of serum from animal | | | | | | | | |
|---|---|---|---|---|---|---|---|---|---|
| | 95011 | 96032 | 95042 | 96058 | | | | | |
| | (Tat | (Tat | (Tat | (Tat | 96061 | 96079 | 96116 | 96122 | 96134 |
| Peptide | Tx) | Tx) | Tx) | Tx) | (Tat Tx) | (Tat) | (Tat) | (Tat) | (Tat) |
| aa 46-65 (SEQ ID NO. 35) | 2.412, | 2.474, | 1.558, | 1.25, | 0.254, | 0.053, | 1.322, | 0.234, | 2.025, |
| (SYGSKKRRQRRRPPQDNQTH) | 2.452 | 2.457 | 1.624 | 1.267 | 0.257 | 0.052 | 1.336 | 0.259 | 2.007 |
| aa 46-60 (SEQ ID NO 13) | 0.867, | 1.678, | 1.169, | 1.142, | 0.331, | 0.196, | 1.052, | 0.723, | 2.279, |
| (SYGSKKRRQRRRPPQ) | 0.865 | 1.664 | 1.098 | 1.138 | 0.317 | 0.182 | 1.012 | 0.743 | 2.318 |
| aa 41-56 (SEQ ID NO. 15) | 0.059, | 0.519, | 0.145, | 0.79, | 0.124, | 0.036, | 0.78, | 0.699, | 0.071, |
| (KALGISYGSKKRRQRR) | 0.062 | 0.533 | 0.145 | 0.796 | 0.105 | 0.032 | 0.76 | 0.648 | 0.145 |

Three animals (96061, 96079, and 96122) had weak responses to this region. For five animals (96032, 95042, 96058, 96116, and 96134), the response to the truncated peptide including aa 46 to 60 was strong, and the response to the peptide including aa 41 to 56 was less. Antisera from these macaques recognize an epitope that includes aa 57 to 60. For animal 95011, the response to aa 46 to 60 was substantially less than the response to aa 46 to 65. In this animal the epitope may be more complex and potentially shifted to the C terminus compared with the other sera.

Overall, Tat toxoid immunization elicited antibody responses to a limited set of linear epitopes in the Tat molecule as shown in FIG. 3B. Aa significant antibody response was not found to the fifth and the sixth peptide sets, representing C-terminal parts of the 86-aa Tat sequence, despite the fact that the endpoint titers were similar to those in animals immunized with unmodified Tat antigen (36).

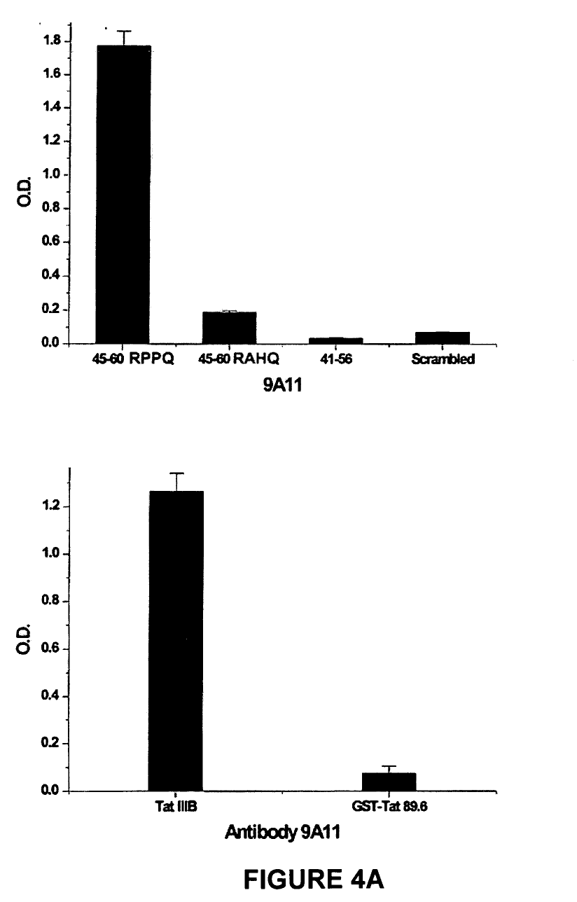

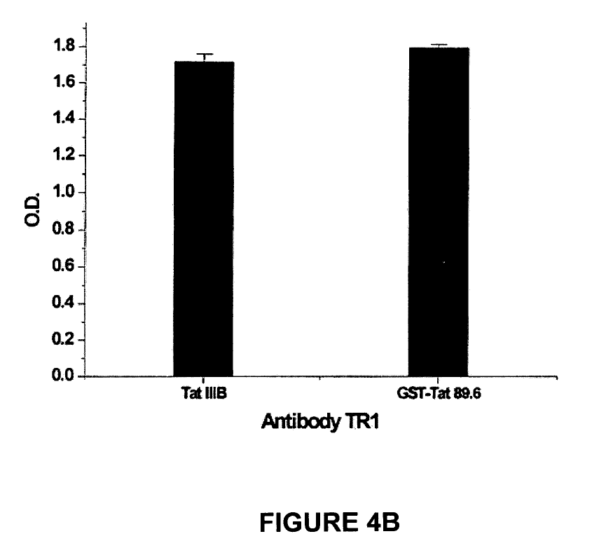

Antibodies discriminate amino acid sequences in Tat aa 57 to 60. The peptide consisting of aa 46 to 60 was used to show the presence of antibodies to the Tat basic domain. In order to define the epitope, a monoclonal antibody was generated against this region. BALB/c mice were immunized with Tat toxoid, and we identified several monoclonal antibodies reacting with the Tat protein. One of the antibodies demonstrated strong reactivity with the basic peptide 46SYGSKKRRQRRRPPQ60 (SEQ ID NO. 13). This antibody reacted poorly with the peptide consisting of aa 41 to 56 (41KALGISYGSKKRRQRR56) (SEQ ID NO. 15), showing that the epitope includes all or part of aa 57 to 60. The antibody bound only weakly with the sequence 46SYGSKKRRQRRRAHQ60, confirming that the 56RPPQ60 sequence is within the epitope. The same pattern of reactivity was found with polyclonal sera from immunized mice (not shown). Monoclonal antibody 9A11 had much better binding to the homologous Tat protein (containing the 56RPPQ60 sequence) than to a related Tat sequence from HIV-1 89.6, which has a 56RAHQ60 sequence, as shown in FIG. 4a. The sequence from aa 57 to 60 is part of a variable epitope located next to the highly conserved basic region sequence 41KALGISYGSKKRRQRR56 (SEQ ID NO. 15).

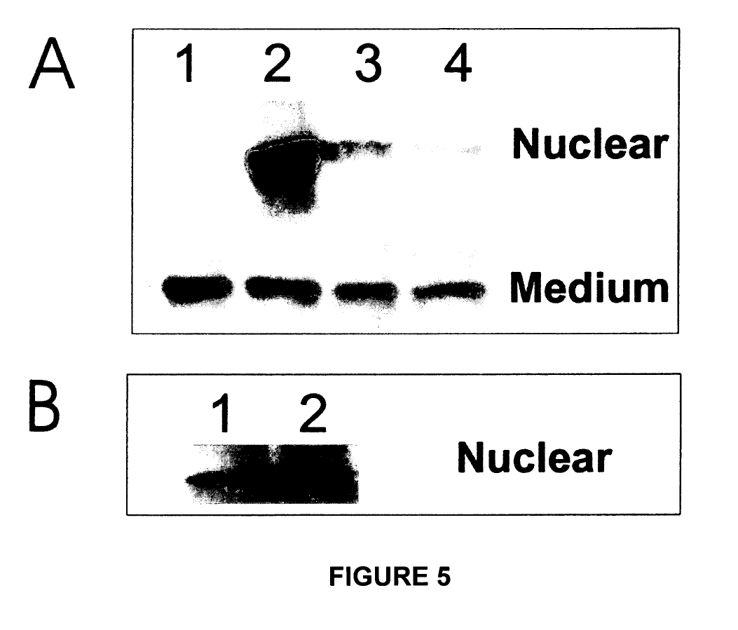

Tat internalization in Jurkat cells. Individual Tat preparations were tested to measure the rate and extent of nuclear accumulation in Jurkat cells as part of developing assays for antibody neutralization of Tat protein. Tat levels in the nucleus and medium were measured by a Western blotting assay as shown in FIG. 5A. There was little change in the concentration of extracellular Tat throughout the study, showing that only a small percentage of the Tat preparation was taken up by Jurkat cells. High levels of nuclear Tat were seen only in the first incubation, indicating that just a fraction of the starting material could enter the cells and travel to the nucleus. On the basis of band intensity in Western blots, the fraction of Tat competent to enter the cell nucleus was roughly tested to be about <5% of the starting material. As a control, Tat was incubated at 37° C. for 90 min and then added to Jurkat cells. The control incubation had little effect on Tat uptake into Jurkat cells as shown in FIG. 5B. The protein appeared to be relatively stable in tissue culture medium and retained a similar, albeit low, capacity for penetrating Jurkat cells.

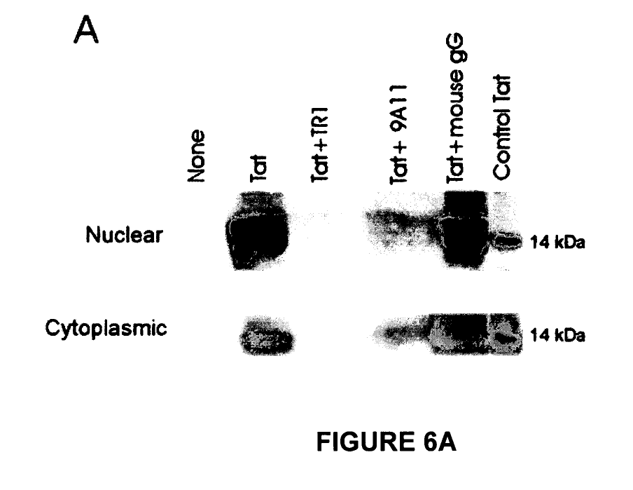

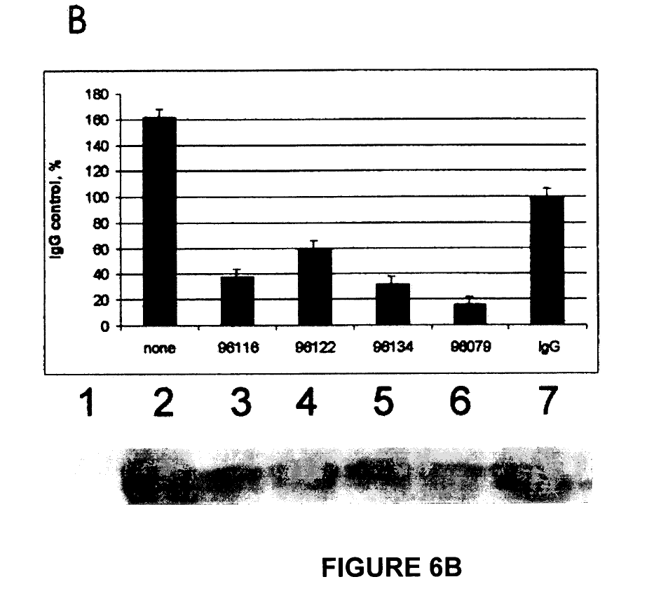

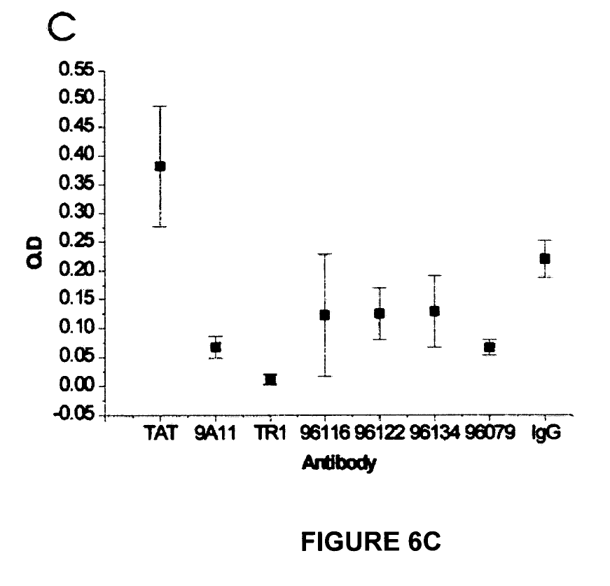

It was found that sera from immunized macaques or mouse monoclonal antibodies blocked Tat uptake by Jurkat cells. Incubation of recombinant Tat with monoclonal anti-Tat antibodies directed against aa 1 to 15 (TR1) or against aa 57 to 60 (9A11), but not incubation with a control antibody, inhibited the accumulation of Tat in nuclear and cytoplasmic fractions of Jurkat cells as shown in FIG. 6A. Substantial inhibition of uptake was also seen with purified immune monkey IgG as shown in FIG. 6B, although high concentrations of control monkey IgG also partly blocked uptake.

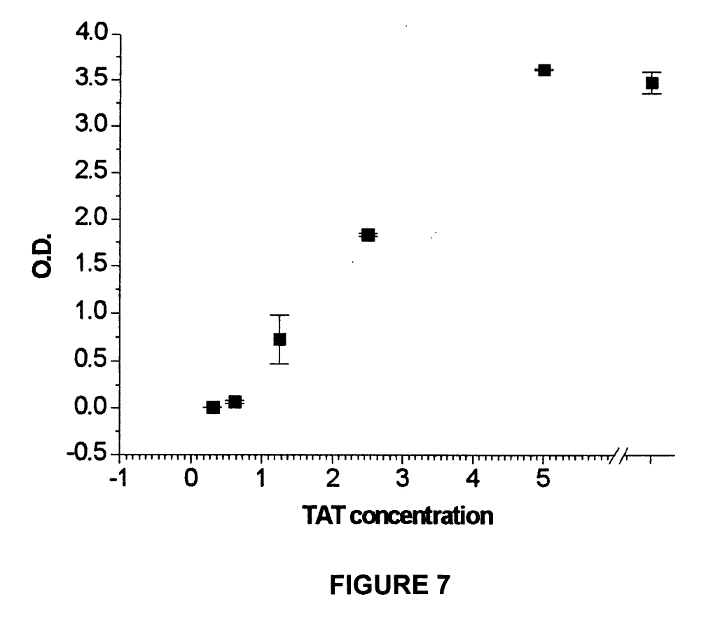

Tat transactivation in HeLa cells. Monoclonal antibodies or purified IgG was tested to determine whether they could neutralize Tat activity in CD4⁺ HeLa cells containing a defective provirus. It was discovered that a normal dose-response curve was first able to be determined for soluble Tat protein and that transactivation with Tat concentrations of around 1 μg/ml could be measured as shown in FIG. 7. For antibody neutralization studies it was important to keep the antigen levels as low as possible.

Monoclonal antibodies 9A11 and TR1 were tested and purified IgG from four macaques that were immunized with Tat protein for the ability to block transactivation. Monoclonal antibodies 9A11 and TR1, which were shown previously to block uptake, strongly neutralized the transactivation activity of Tat protein as shown in FIG. 6C, giving a seven fold reduction in optical density for the p24 antigen capture ELISA. Purified IgG from macaque 96079 also strongly neutralized Tat activity.

In order to characterize the antibodies elicited by immunization, an array was developed of overlapping peptides representing the first 86 aa from three lade B Tat sequences, the lade C consensus sequence, and one individual clade C Tat sequence. The array was interrogated with antisera from four Tat-immunized and five Tat toxoid-immunized macaques; all macaques exhibited Tat-binding antibody titers of between 5,000 and 64,000. Sera from Tat-immunized macaques reacted with several regions of Tat, and there was little discrimination among lade B and lade C sequences. The antibody response to Tat toxoid was more restricted, with substantial recognition of only two Tat regions (amino terminus and basic domain), and the antibodies to the basic domain recognized only one of the clade B sequences. Thus, antibodies raised against Tat recognized epitopes that were generally conserved among clade B and lade C Tat sequences, while antibodies present after Tat toxoid immunization recognized only two main epitopes, and one of these (including aa 57 to 60) was present in only one of the lade B sequences and in neither of the lade C sequences. The same 57RPPQ60 sequence was present in both the Tat and Tat toxoid antigens. Importantly, the chemical modifications in Tat toxoid occurred in the cysteine-rich domain (aa 21 to 40), yet they affected antibody responses to distant sequences at aa 57 to 60.

The epitope 57RPPQ60 was recognized by sera from immunized macaques and monoclonal antibodies from immunized mice. A monoclonal antibody against this sequence reacted strongly with a homologous Tat preparation and with peptides containing the RPPQ sequence. The same antibody did not react with a Tat preparation or peptides having the sequence RAHQ at this position. The sequences RAHQ and RAPQ are common at positions 57 to 60 for lade B Tat proteins, while clade C viruses tend to have the sequence SAPQ at this same site. Recognition of the 57RPPQ60 epitope may be important for clinical studies of Tat immunity. This sequence is infrequent among lade B isolates and, heretofore, has never been reported to be present in a lade C isolate.

Monoclonal anti-Tat antibodies directed against aa 1 to 15 (TR1) or against aa 57 to 60 (9A11) inhibited the accumulation of extracellular Tat in the cytoplasm and nuclei of Jurkat cells as shown in FIG. 6A. Surprisingly, the amino-terminus-specific antibody TR1 showed the strongest inhibition of Tat uptake. These data show a requirement for the amino terminus in Tat internalization and argue against the idea that basic sequences are sufficient for internalization of the intact Tat molecule (18, 43, 50) even though they function as protein transduction domains in chimeric proteins.

It was observed that <5% of the Tat preparation used here was competent to enter cells. Even though Tat was added at a concentration of 1 μg/ml to measure uptake or transactivation, the active fraction was present at less than 5% or 50 ng/ml, a concentration comparable to what is used commonly to measure biological activity of cytokines and chemokines. The low specific activity of Tat preparations necessitated the addition of large amounts of Tat in transactivation assays. The presence of large amounts of inactive protein reduces the ability of monoclonal antibodies or polyclonal IgG to block uptake or to neutralize the transactivation activity.

The mechanisms for Tat neutralization were indistinguishable from those for the inhibition of Tat uptake. However, for neutralization of Tat uptake and viral transactivation, it was sufficient to have antibodies against the amino terminus of the Tat protein. Apparently, the basic region, or transduction domain, that mediates uptake of many other proteins (18, 43, 45) is not sufficient for Tat uptake, and portions of the amino terminus may also be required. This result, combined with the observation that only a small fraction of Tat molecules are competent for internalization, argues that a specific conformer of Tat carries the highest activity. Such a conclusion would not be apparent from the published structural studies, where Tat is reported to have little secondary or tertiary structure (7, 24).

### REFERENCES

b 1. Albini, A., R. Benelli, D. Giunciuglio, T. Cai, G. Mariani, S. Ferrini, and D. M. Noonan. 1998. Identification of a novel domain of HIV tat involved in monocyte chemotaxis. J. Biol. Chem. 273:15895-15900.
2. Albini, A., S. Ferrini, R. Benelli, S. Sforzini, D. Giunciuglio, M. G. Aluigi, A. E. Proudfoot, S. Alouani, T. N. Wells, G. Mariani, R. L. Rabin, J. M. Farber, and D. M. Noonan. 1998. HIV-1 Tat protein mimicry of chemokines. Proc. Nati. Acad. Sci. USA 95:13153-13158.
3. Allen, T. M., L. Mortara, B. R. Mothe, M. Liebl, P. Jing, B. Calore, M. Piekarczyk, R. Ruddersdorf, D. H. O'Connor, X. Wang, C. Wang, D. B. Allison, J. D. Altman, A. Sette, R. C. Desrosiers, G. Sutter, and D. I. Watkins. 2002. Tat-vaccinated macaques do not control simian immunodeficiency virus SIVmac239 replication. J. Virol. 76:4108-4112.
4. Badou, A., Y. Bennasser, M. Moreau, C. Leclerc, M. Benkirane, and E. Bahraoui. 2000. Tat protein of human immunodeficiency virus type 1 induces interleukiamino 10 in human peripheral blood monocytes: implication of protein kinase C-dependent pathway. J. Virol. 74:10551-10562.
5. Barillari, G., R. Gendelman, R. C. Gallo, and B. Ensoli. 1993. The Tat protein of human immunodeficiency virus type 1, a growth factor for AIDS Kaposi sarcoma and cytokine-activated vascular cells, induces adhesion of the same cell types by using integrin receptors recognizing the RGD amino acid sequence. Proc. Natl. Acad. Sci. USA 90:7941-7945.
6. Bartz, S. R., and M. Emerman. 1999. Human immunodeficiency virus type 1 Tat induces apoptosis and increases sensitivity to apoptotic signals by up-regulating FLICE/caspase-8. J. Virol. 73:1956-1963.
7. Bayer, P., M. Kraft, A. Ejchart, M. Westendorp, R. Frank, and P. Rosch. 1995. Structural studies of HIV-1 Tat protein. J. Mol. Biol. 247:529-535.
8. Benjouad, A., K. Mabrouk, M. Moulard, J. C. Gluckman, H. Rochat, J. Van Rietschoten, and J. M. Sabatier. 1993. Cytotoxic effect on lymphocytes of Tat from human immunodeficiency virus (HIV-1). FEBS Lett. 319:119-124.
9. Boykins, R. A., R. Mahieux, U. T. Shankavaram, Y. S. Gho, S. F. Lee, I. K. Hewlett, L. M. Wahl, H. K. Kleinman, J. N. Brady, K. M. Yamada, and S. Dhawan. 1999. Cutting edge: a short polypeptide domain of HIV-1-Tat protein mediates pathogenesis. J. Immunol. 163:15-20.
10. Brake, D. A., C. Debouck, and G. Biesecker. 1990. Identification of an Arg-Gly-Asp (RGD) cell adhesion site in human immunodeficiency virus type I transactivation protein, tat. J. Cell Biol. 111:1275-1281.
11. Brake, D. A., J. Goudsmit, W. J. Krone, P. Schammel, N. Appleby, R. H. Meloen, and C. Debouck. 1990. Characterization of murine monoclonal antibodies to the Tat protein from human immunodeficiency virus type 1. J. Virol. 64:962-965
12. Cafaro, A., A. Caputo, C. Fracasso, M. T. Maggiorella, D. Goletti, S. Baroncelli, M. Pace, L. Sernicola, M. L. Koanga-Mogtomo, M. Betti, A. Borsetti, R. Belli, L. Akerblom, F. Corrias, S. Butto, J. Heeney, P. Verani, F. Titti, and B. Ensoli. 1999. Control of SHIV-89.6P-infection of cynomolgus monkeys by HTV-1 Tat protein vaccine. Nat. Med. 5:643-650.
13. Cohen, S. S., C. Li, L. Ding, Y. Cao, A. B. Pardee, E. M. Shevach, and D. I. Cohen. 1999. Pronounced acute immunosuppression in vivo mediated by HIV Tat challenge. Proc. Natl. Acad. Sci. USA 96:10842-10847.
14. Cordingley, M. G., R. L. LaFemina, P. L. Callahan, J. H. Condra, V. V. Sardana, D. J. Graham, T. M. Nguyen, K. LeGrow, L. Gotlib, A. J. Schlabach, et al. 1990. Sequence-specific interaction of Tat protein and Tat peptides with the transactivatioamino responsive sequence element of human immunodeficiency virus type I in vitro. Proc. Natl. Acad. Sci. USA 87:8985-8989
15. Delling, U., S. Roy, M. Sumner-Smith, R. Barnett, L. Reid, C. A. Rosen, and N. Sonenberg. 1991. The number of positively charged amino acids in the basic domain of Tat is critical for trans-activation and complex formation with TAR RNA. Proc. Natl. Acad. Sci. USA 88:6234-6238.
16. de Paulis, A., R. De Palma, L. Di Gioia, M. Carfora, N. Prevete, G. Tosi, R. S. Accolla, and G. Marone. 2000. Tat protein is an HIV-1-encoded beta-chemokine homolog that promotes migration and up-regulates CCR3 expression on human Fc epsilon RI+ cells. J. Immunol. 165:7171-7179.
17. Ensoli, B., L. Buonaguro, G. Barillari, V. Fiorelli, R. Gendelman, R. A. Morgan, P. Wingfield, and R. C. Gallo. 1993. Release, uptake, and effects of extracellular human immunodeficiency virus type I Tat protein on cell growth and viral transactivation. J. Virol. 67:277-287.
18. Fawell, S., J. Seery, Y. Daikh, C. Moore, L. L. Chen, B. Pepinsky, and J. Barsoum. 1994. Tat-mediated delivery of heterologous proteins into cells. Proc. NatI. Acad. Sci. USA 91:664-668
19. Galfre, G., and C. Milstein. 1981. Preparation of monoclonal antibodies: strategies and procedures. Methods Enzymol. 73:3-46.
20. Gallo, R. C. 1999. Tat as one key to HIV-induced immune pathogenesis and Tat (correction of Pat) toxoid as an important component of a vaccine. Proc. Natl. Acad. Sci. USA 96:8324-8326.
21. Goldstein, G. 1996. HIV-1 Tat protein as a potential AIDS vaccine. Nat. Med. 2:960-964.
22. Goldstein, G., K. Manson, G. Tribbick, and R. Smith. 2000. Minimization of chronic plasma viremia in rhesus macaques immunized with synthetic HIV-1 Tat peptides and infected with a chimeric simian/human immunodeficiency virus (SHIV33). Vaccine 18:2789-2795
23. Goldstein, G., G. Tribbick, and K. Manson. 2001. Two B cell epitopes of HIV-1 Tat protein have limited antigenic polymorphism in geographically diverse HIV-1 strains. Vaccine 19:1738-1746.
24. Gregoire, C., J. M. Peloponese, Jr., D. Esquieu, S. Opi, G. Campbell, M. Solomiac, E. Lebrun, J. Lebreton, and E. P. Loret. 2001. Homonuclear (1)H-NMR assignment and structural characterization of human immunodeficiency virus type 1 Tat Mal protein. Biopolymers 62:324-335.
25. Gringeri, A., E. Santagostino, M. Muca-Perja, P. M. Mannucci, J. F. Zagury, B. Bizzini, A. Lachgar, M. Carcagno, J. Rappaport, M. Criscuolo, W. Blattner, A. Burny, R. C. Gallo, and D. Zagury. 1998. Safety and immunogenicity of HIV-1 Tat toxoid in immunocompromised HfV-1-infected patients. J. Hum. Virol. 1:293-298.
26. Gutheil, W. G., M. Subramanyam, G. R. Flentke, D. G. Sanford, E. Munoz, B. T. Huber, and W. W. Bachovchin. 1994. Human immunodeficiency virus 1 Tat binds to dipeptidyl aminopeptidase IV (CD26): a possible mechanism for Tat's immunosuppressive activity. Proc. Natl. Acad. Sci. USA 91:6594-6598.
27. Hall, T. A. 1999. BioEdit: a user friendly biological sequence alignment editor and analysis program for Windows 95/98/NT. Nucleic Acids Symp. Ser. 41:95-98.
28. Huang, L., I. Bosch, W. Hofmann, J. Sodroski, and A. B. Pardee. 1998. Tat protein induces human immunodeficiency virus type 1 (HIV-1) coreceptors and promotes infection with both macrophage-tropic and T-lymphotropic HIV-1 strains. J. Virol. 72:8952-8960.
29. Ito, M., T. Ishida, L. He, F. Tanabe, Y. Rongge, Y. Miyakawa, and H. Terunuma. 1998. HIV type I Tat protein inhibits interleukin 12 production by human peripheral blood mononuclear cells. AIDS Res. Hum. Retroviruses 14:845-849.
30. Jeang, K. T., H. Xiao, and E. A. Rich. 1999. Multifaceted activities of the HIV-1 transactivator of transcription, Tat. J. Biol. Chem. 274:28837-28840.
31. Katsikis, P. D., M. E. Garcia-Ojeda, J. F. Torres-Roca, D. R. Greenwald, and L. A. Herzenberg. 1997. HIV type I Tat protein enhances activatioamino but not Fas (CD95)-induced peripheral blood T cell apoptosis in healthy individuals. Int. Immunol. 9:835-841.
32. Lambert, J. 1998. Tat toxoid: its potential role as an HIV vaccine. J. Hum. Virol. 1:249-250.
33. Le Buanec, H., and B. Bizzini. 2000. Procedures for preparing biologically inactive, but immunogenic HIV-I Tat protein (Tat toxoid) for human use. Biomed. Pharmacother. 54:41-44.
34. Macho, A., M. A. Calzado, L. Jimenez-Reina, E. Ceballos, J. Leon, and E. Munoz. 1999. Susceptibility of HIV-1-TAT transfected cells to undergo apoptosis. Biochemical mechanisms. Oncogene 18:7543-7551.
35. Mann, D. A., and A. D. Frankel. 1991. Endocytosis and targeting of exogenous WIV-I Tat protein. EMBO J. 10:1733-1739
36. Pauza, C. D., P. Trivedi, M. Wallace, T. J. Ruckwardt, H. Le Buanec, W. Lu, B. Bizzini, A. Burny, D. Zagury, and R. C. Gallo. 2000. Vaccination with tat toxoid attenuates disease in simian/HIV-challenged macaques. Proc. Natl. Acad. Sci. USA 97:3515-3519.
37. Philippon, V., C. Vellutini, D. Gambarelli, G. Harkiss, G. Arbuthnott, D. Metzger, R. Roubin, and P. Filippi. 1994. The basic domain of the lentiviral Tat protein is responsible for damages in mouse brain: involvement of cytokines. Virology 205:519-529.
38. Re, M. C., G. Furlini, M. Vignoli, E. Ramazzotti, G. Roderigo, V. De Rosa, G. Zauli, S. Lolli, S. Capitani, and M. La Placa. 1995. Effect of antibody to HIV-1 Tat protein on viral replication in vitro and progression of HIV-1 disease in vivo. J. Acquir. Immune Defic. Syndr. Hum. Retrovirol. 10:408-416.
39. Re, M. C., M. Vignoli, G. Furlini, D. Gibellini, V. Colangeli, F. Vitone, and M. La Placa. 2001. Antibodies against full-length Tat protein and some low-molecular-weight Tat-peptides correlate with low or undetectable viral load in HIV-1 seropositive patients. J. Clin. Virol. 21:81-89.
40. Rodman, T. C., J. D. Lutton, S. Jiang, H. B. Al-Kouatly, and R. Winston. 2001. Circulating natural IgM antibodies and their corresponding human cord blood cell-derived Mabs specifically combat the Tat protein of HIV. Exp. Hematol. 29:1004-1009.
41. Rodman, T. C., S. E. To, H. Hashish, and K. Manchester. 1993. Epitopes for natural antibodies of human immunodeficiency virus (HIV)-negative (normal) and HIV-positive sera are coincident with two key functional sequences of HIV Tat protein. Proc. Natl. Acad. Sci. USA 90:7719-7723.
42. Ruben, S., A. Perkins, R. Purcell, K. Joung, R. Sia, R. Burghoff, W. A. Haseltine, and C. A. Rosen. 1989. Structural and functional characterization of human immunodeficiency virus Tat protein. J. Virol. 63:1-8.
43. Rusnati, M., G. Tulipano, C. Urbinati, E. Tanghetti, R. Giuliani, M. Giacca, M. Ciomei, A. Corallini, and M. Presta. 1998. The basic domain in HIV-1 Tat protein as a target for polysulfonated hepariamino mimicking extracellular Tat antagonists. J. Biol. Chem. 273:16027-16037.
44. Sadaie, M. R., T. Benter, and F. Wong-Staal. 1988. Site-directed mutagenesis of two trans-regulatory genes (tat-III,trs) of HIV-1. Science 239:910-913.
45. Schwarze, S. R., A. Ho, A. Vocero-Akbani, and S. F. Dowdy. 1999. In vivo protein transduction: delivery of a biologically active protein into the mouse. Science 285:1569-1572.
46. Secchiero, P., D. Zella, S. Capitani, R. C. Gallo, and G. Zauli. 1999. Extracellular HIV-1 tat protein up-regulates the expression of surface CXC-chemokine receptor 4 in resting CD4+T cells. J. Immunol. 162:2427-2431.
47. Secchiero, P., D. Zella, S. Curreli, P. Mirandola, S. Capitani, R. C. Gallo, and G. Zauli. 2000. Pivotal role of cyclic nucleoside phosphodiesterase 4 in Tat-mediated CD4+T cell hyperactivation and HIV type 1 replication. Proc. NatI. Acad. Sci. USA 97:14620-14625.
48. Silvera, P., M. W. Richardson, J. Greenhouse, J. Yalley-Ogunro, N. Shaw, J. Mirchandani, K. Khalili, J. F. Zagury, M. G. Lewis, and J. Rappaport. 2002. Outcome of simiaamino human immunodeficiency virus strain 89.6p challenge following vaccination of rhesus macaques with human immunodeficiency virus Tat protein. J. Virol. 76:3800-3809.
49. Subramanyam, M., W. G. Gutheil, W. W. Bachovchin, and B. T. Huber. 1993. Mechanism of HIV-1 Tat induced inhibition of antigeamino specific T cell responsiveness. J. Immunol. 150:2544-2553.
50. Tyagi, M., M. Rusnati, M. Presta, and M. Giacca. 2001. Internalization of HIV-1 tat requires cell surface heparan sulfate proteoglycans. J. Biol. Chem. 276:3254-3261.
51. van Baalen, C. A., 0. Pontesilli, R. C. Huisman, A. M. Geretti, M. R. Klein, F. de Wolf, F. Miedema, R. A. Gruters, and A. D. Osterhaus. 1997. Human immunodeficiency virus type 1 Rev- and Tat-specific cytotoxic T lymphocyte frequencies inversely correlate with rapid progression to AIDS. J. Gen Virol. 78:1913-1918.
52. Viscidi, R. P., K. Mayur, H. M. Lederman, and A. D. Frankel. 1989. Inhibition of antigeamino induced lymphocyte proliferation by Tat protein from HIV-1. Science 246:1606-1608.
53. Vives, E., P. Brodin, and B. Lebleu. 1997. A truncated HIV-1 Tat protein basic domain rapidly translocates through the plasma membrane and accumulates in the cell nucleus. J. Biol. Chem. 272:16010-16017.
54. Weeks, K. M., and D. M. Crothers. 1991. RNA recognition by Tat-derived peptides: interaction in the major groove? Cell 66:577-588.
55. Weiss, J. M., A. Nath, E. 0. Major, and J. W. Berman. 1999. HIV-I Tat induces monocyte chemoattractant proteiamino 1-mediated monocyte transmigration across a model of the human blood-brain barrier and up-regulates CCR5 expression on human monocytes. J. Immunol. 163:2953-2959.
56. Westendorp, M. O., R. Frank, C. Ochsenbauer, K. Stricker, J. Dhein, H. Walczak, K. M. Debatin, and P. H. Krammer. 1995. Sensitization of T cells to CD95-mediated apoptosis by HIV-1 Tat and gp120. Nature 375:497-500.
57. Wrenger, S., T. Hoffmann, J. Faust, C. Mrestani-Klaus, W. Brandt, K. Neubert, M. Kraft, S. Olek, R. Frank, S. Ansorge, and D. Reinhold. 1997. The amino terminal structure of HIV-1 Tat is required for suppression of CD26-dependent T cell growth. J. Biol. Chem. 272:30283-30288.
58. Zagury, D., A. Lachgar, V. Chams, L. S. Fall, J. Bernard, J. F. Zagury, B. Bizzini, A. Gringeri, E. Santagostino, J. Rappaport, M. Feldman, A. Burny, and R. C. Gallo. 1998. Interferon alpha and Tat involvement in the immunosuppression of uninfected T cells and C—C chemokine decline in AIDS. Proc. NatI. Acad. Sci. USA 95:3851-3856.
59. Zagury, J. F., A. Sill, W. Blattner, A. Lachgar, H. Le Buanec, M. Richardson, J. Rappaport, H. Hendel, B. Bizzini, A. Gringeri, M. Carcagno, M. Criscuolo, A. Bumy, R. C. Gallo, and D. Zagury. 1998. Antibodies to the HIV-1 Tat protein correlated with nonprogression to AIDS: a rationale for the use of Tat toxoid as an HIV-1 vaccine. J. Hum. Virol. 1:282-292.
60. Zhang, M., X. Li, X. Pang, L. Ding, O. Wood, K. Clouse, I. Hewlett, and A. I. Dayton. 2001. Identification of a potential HIV-induced source of bystander-mediated apoptosis in T cells: upregulation of trail in primary human macrophages by HIV-1 tat. J. Biomed. Sci. 8:290-296.

1-2. (canceled)

3. A therapeutic composition comprising at least one peptide having an amino acid sequence consisting of an amino acid sequence selected from sequences set forth in SEQ ID NO: 1, SEQ ID NO:2, SEQ ID NO: 3, SEQ ID NO: 4, SEQ ID NO: 5 or SEQ ID NO: 6.

4. The therapeutic composition according to claim 3, wherein the amino acid sequence is conjugated to a carrier protein.

5. The therapeutic composition according to claim 4, wherein the carrier protein is a viral carrier protein.

6. The therapeutic composition according to claim 5, wherein the viral carrier protein is selected from the group consisting of gag, env, nef and fragments thereof.

7. The therapeutic composition according to claim 3, further comprising a pharmaceutically acceptable carrier.

8. A therapeutic vaccine comprising at least one Tat linear epitope peptide comprising from about 15 to about 21 amino acid residues from the amino terminus region of HIV Tat, wherein the amino acid sequence comprises at least amino acid residue 1, 7 and 12.

9. The therapeutic vaccine according to claim 8, wherein the Tat linear epitope peptide is conjugated to a carrier protein.

10. The therapeutic vaccine according to claim 9, wherein the carrier protein is ovalbumin or a viral carrier protein.

11. The therapeutic vaccine according to claim 10, wherein the viral carrier protein is gag, env, nef or fragments thereof.

12. A method to induce production of neutralizing Tat antibodies that inhibit internalization of Tat into T-cells, the method comprising:
administering to a subject a effective amount of a vaccine to induce production of neutralizing Tat antibodies, the vaccine comprising at least one peptide having at least 15 amino acid residues from the amino terminus region of Tat conjugated to a viral carrier protein, wherein the amino acid sequence comprises at least amino acid residue 1, 7 and 12.

13. The method according to claim 12, wherein the peptide is selected from sequences set forth in SEQ ID NO: 1, SEQ ID NO:2, SEQ ID NO: 3, SEQ ID NO: 4, SEQ ID NO: 5 or SEQ ID NO: 6.

14. The method according to claim 13, wherein the viral carrier protein is env, gag, nef or fragments thereof.

15. The method according to claim 14, wherein the vaccine is administered contemporaneously with an antiviral agent.

16. The method according to claim 15, wherein the antiviral agent is selected from nucleoside RT inhibitors, CCR5 inhibitors/antagonists, viral entry inhibitors or their functional analogs.

17-21. (canceled)

22. A polynucleotide sequence comprising a nucleotide sequence encoding a peptide having at least about 15 to about 21 amino acid residues from the amino terminus region of HIV Tat, wherein the peptide comprises at least amino acid residue 1, 7 and 12.

23. The polynucleotide sequence according to claim 22, wherein the nucleotide sequence is selected from sequences set forth in SEQ ID NO: 7, SEQ ID NO: 8, SEQ ID NO: 9, SEQ ID NO: 10, SEQ ID NO: 11 or SEQ ID NO: 12.

24. The polynucleotide sequence according to claim 23, wherein the nucleotide sequence encoding the peptide is linked to a nucleotide sequence encoding a viral carrier protein.

25. The polynucleotide sequence according to claim 24, wherein the viral carrier protein is gag.

26. An expression vector comprising the polynucleotide sequence according to claim 22.

27. An expression vector comprising the polynucleotide sequence according to claim 23.

28. An expression vector comprising the polynucleotide sequence according to claim 25.

29.-31. (canceled)

32. A method of expressing a Tat amino terminus linear epitope peptide comprising the steps of:
(a) transfecting a recombinant host cell with a polynucleotide according to claim 22; (b) culturing the host cell under conditions sufficient for expression of the Tat amino terminus linear epitope peptide; (c) recovering the Tat amino terminus linear epitope peptide.

33-36. (canceled)

37. An antibody immunoreactive with a Tat amino terminus linear epitope peptide according to claim 3 4.

38-39. (canceled)

40. A method of producing an antibody that is immunoreactive with a Tat amino terminus linear epitope peptide according to claim 8 comprising the steps of:
(a) introducing a Tat amino terminus linear epitope peptide according to claim 1 into a live animal subject; and
(b) recovering the antibody

41-45. (canceled)
